(19) United States
(12) Patent Application Publication
(10) Pub. No.: US 2023/0181688 A1
(43) Pub. Date: Jun. 15, 2023

(54) METHODS FOR TREATING PLASMA PROTEIN IMBALANCES OR DEPLETION

(71) Applicants: Ohio State Innovation Foundation, Columbus, OH (US); THE REGENTS OF THE UNIVERSITY OF CALIFORNIA, Oakland, CA (US)

(72) Inventors: Ivan SUSIN PIRES, Cambridge, MA (US); Andre PALMER, Bexley, OH (US); Pedro J. CABRALES AREVALO, La Mesa, CA (US)

(21) Appl. No.: 17/926,479

(22) PCT Filed: Mar. 22, 2021
(86) PCT No.: PCT/US2021/023441

§ 371 Date: Nov. 18, 2022

Related U.S. Application Data
(60) Provisional application No. 63/027,556, filed on May 20, 2020.

(51) Int. Cl. A61K 38/17; A61K 38/40; A61K 38/38; A61K 38/44; A61P 7/04

(52) U.S. Cl. CPC A61K 38/1722; A61K 38/40; A61K 38/44; A61K 38/385; A61P 7/04; C12Y 116/03001

(57) ABSTRACT

The present disclosure provides methods of treating plasma protein imbalances or depletion (e.g., plasma protein imbalances or depletion caused by hemorrhagic shock or other clinical conditions), in particular by administering protein compositions derived from plasma or plasma isolates.

### CROSS-REFERENCE TO RELATED APPLICATIONS

This application claims the benefit of priority to U.S. Provisional Application No. 63/027,556, filed May 20, 2020, the disclosure of which is incorporated herein by reference in its entirety.

### STATEMENT REGARDING FEDERALLY SPONSORED RESEARCH OR DEVELOPMENT

This invention was made with Government Support under Grant No. R01EB021926, Grant No. R01HL126945, and Grant No. R01HL138116, all awarded by the National Institutes of Health. The Government has certain rights in the invention.

### TECHNICAL FIELD

This disclosure relates to methods for the treatment of plasma protein imbalances or depletion (e.g., plasma protein imbalances or depletion caused by hemorrhagic shock or other clinical conditions), and more particularly to methods comprising administration of protein compositions for such treatment.

### BACKGROUND

Hemorrhagic shock (HS) is a major cause or morbidity and mortality after severe trauma. Commonly-used fluids for resuscitation from HS include crystalloids (e.g., saline), colloids such as human serum albumin (HSA), plasma, or blood. Unfortunately, blood products generally have short ex vivo storage lifetimes and must be stored under stringent environmental conditions. Furthermore, blood is not easily transportable in austere environments. Plasma, crystalloids, and HSA can all be lyophilized, greatly improving storage and transportation of the therapeutics to the point-of-care.

Unfortunately, crystalloid solutions may cause deleterious side effects upon transfusion by eliciting an immune response (See Krausz, M. M. Initial resuscitation of hemorrhagic shock. World Journal of Emergency Surgery 1, (2006)). Colloid solutions can be expensive and can cause fluid imbalances and may even exacerbate loss of extracellular fluid volume. However, compared to crystalloids, colloids have the benefit of requiring less fluid volume for resuscitation. In addition, the large volume of transfused crystalloids can cause tissue edema, an increase in the incidence of abdominal compartment syndrome, and hyperchloremic metabolic acidosis (see Bougie, A., Harrois, A. and Duranteau, J. Resuscitative strategies in traumatic hemorrhagic shock. Annals of Intensive Care 3, 1-9 (2013)). Recently, lyophilized plasma has gained attention for use in treating HS. However, the availability of plasma and issues associated with cross-matching blood group antigens can restrict its use.

HS or trauma is often accompanied by hemolysis. Hemolysis is characterized by the rupture of red blood cells, thus releasing free hemoglobin, free heme and free iron into the blood stream. These components of red blood cells are toxic and rely on the body’s natural supply of plasma scavenger proteins such as haptoglobin, hemopexin, and transferrin to detoxify them. This scavenging of iron-based toxic molecules released during hemolysis also may reduce the severity of bacterial infections, possibly helping to prevent sepsis. During HS, however, these scavenger proteins are depleted both due to fluid loss and the presence of excess hemoglobin, heme, and iron in circulation as compared to normal levels.

Thus, there is a clear need for new therapeutics for the treatment of hemorrhagic shock.

### SUMMARY

The present disclosure provides methods for treating plasma protein imbalances or depletion using protein compositions as described herein. The plasma protein imbalance or depletion can be caused by, for example, hemorrhagic shock, burns, surgery, organ transplantation, a hypovolemic state, a hypervolemic state, sepsis, trauma, subcutaneous trauma, kidney dialysis, traumatic brain injury, traumatic brain injury combined with hemorrhagic shock, a coagulation disorder, or any combination thereof. In some embodiments, the plasma protein imbalance or depletion requires a plasma expander or a blood volume replacement. The protein compositions as used in the methods described herein provide an effective, inexpensive, and transportable fluid for treating such plasma protein imbalances or depletion.

Thus in one aspect, a method for treating a plasma protein imbalance or depletion (e.g., a plasma protein imbalance or depletion caused by hemorrhagic shock) is provided comprising administering a therapeutically effective amount of a protein composition comprising:
from 5% to 99% by weight haptoglobin, based on the total weight of all proteins in the protein composition; and
from 1% to 95% by weight transferrin, based on the total weight of all proteins in the protein composition.

In some embodiments, the composition as used herein is substantially free of immunogenic proteins, for example antibodies.

In some embodiments, the composition as used herein comprises from 5% to 60% by weight haptoglobin, for example from 5% to 25% by weight haptoglobin, based on the total weight of all proteins in the composition. In some embodiments, the haptoglobin as found in the compositions used herein has an average molecular weight of from 80 kDa to 1,000 kDa, for example from 80 kDa to 500 kDa. In some embodiments, the haptoglobin is characterized by having residual hemoglobin as characterized by UV-visible spectroscopy of the Soret peak ranging from 402-407 nm.

In some embodiments, the composition as used herein comprises from 1% to 60% by weight transferrin, for example from 30% to 40% by weight transferrin, based on the total weight of all proteins in the composition.

In some embodiments, the composition as used herein further comprises from 1% to 75% by weight hemopexin, for example from 5% to 40% by weight or from 1% to 10% by weight hemopexin, based on the total weight of all proteins in the composition.

In some embodiments, the composition as used herein further comprises from 1% to 70% by weight albumin, for example from 5% to 30% by weight or from 30% to 50% by weight albumin, based on the total weight of all proteins in the composition. In some embodiments, the albumin comprises polymeric albumin.

In some embodiments, the composition as used herein further comprises an additional protein selected from vitamin-D binding protein, ceruloplasmin, or a combination thereof.

In some embodiments, the composition as used herein comprises:
from 5% to 15% by weight haptoglobin, based on the total weight of all proteins in the composition;
from 30% to 50% by weight albumin, based on the total weight of all proteins in the composition;
from 1% to 10% by weight hemopexin, based on the total weight of all proteins in the composition; and
from 30% to 40% by weight transferrin, based on the total weight of all proteins in the composition;
wherein the composition is substantially free of immunogenic proteins. In some embodiments, the composition further comprises from 5% to 1 5% by weight vitamin-D binding protein, ceruloplasmin, or a combination thereof, based on the total weight of all proteins in the composition.

In some embodiments, the plasma protein imbalance or depletion is prophylactically treated (e.g., the compositions described herein are administered to a subject prophylactically).

In some embodiments, the composition is used as an extracorporeal priming fluid.

In some embodiments, the composition is used as a plasma substitute for artificial blood substitutes, such as a hemoglobin-based oxygen carrier.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a representative depiction of the process used to purify haptoglobin (Hp) (Stages 2 and 3), and the protein cocktail described herein (Stage 4). HMW = high MW Hp fraction. LMW =low MW Hp fraction. Arrows indicate the direction of flow.

FIGS. 2A-2D provides representative examples of hemoglobin (Hb) (FIG. 2A), iron (FIG. 2B), total heme (FIG. 2C) and hemopexin (Hpx) heme (FIG. 2D) binding assays to determine the binding capacity of Hp, transferrin (Tf), human serum albumin + Hpx (HSA+Hpx), and Hpx respectively. hHSA = heme-albumin.

FIGS. 3A-3C shows an SDS-PAGE of a representative batch of the protein cocktail under non-reduced (FIG. 3A) and reduced (FIG. 3B) conditions and top ten identified proteins from trypsin digest mass spectrometry of a representative batch of the protein scavenging cocktail (FIG. 3C). Abbreviations: human serum albumin, HSA; transferrin, Tf; haptoglobin, Hp; ceruloplasmin, Cp; vitamin-D binding protein, VDB; hemopexin, Hpx; haptoglobin-related protein, Hpr; immunoglobulin gamma 1 heavy chain, IgGlHC; α-1-B glycoprotein, A1BG; immunoglobulin kappa constant, IgkC.

FIGS. 4A-4D shows MALDI-TOF mass spectral analysis of the protein cocktail under (FIG. 4A) non-reduced and (FIG. 4B) reduced conditions. Peaks denote the mass to charge ratio in m/z. (FIG. 4C) Comparison of protein cocktail with human serum albumin (HSA) under non-reduced conditions denoting the presence of common peaks in the two samples. (FIG. 4D) Comparison of protein cocktail with haptoglobin 2-1, 2-2 mixture (Hp) under reduced conditions resulted in common peaks in the two samples confirming the presence of Hp in the cocktail. Abbreviations: human serum albumin, HSA; transferrin, Tf; haptoglobin, Hp; hemopexin, Hpx; transthyretin, TTR; α-1 antitrypsin, AAT; α-1 antichymotrypsin, α1AC; α chain hemoglobin, α-Hb; α1 chain haptoglobin, α1-Hp; α2 chain haptoglobin, α2-Hp.

FIGS. 5A and 5B presents some biophysical properties of the protein cocktail. (FIG. 5A) Colloidal osmotic pressure (COP). (FIG. 5B) Viscosity.

FIGS. 6A and 6B shows RBC aggregation tests with the protein cocktail. (FIG. 6A) Representative images of RBCs mixed with HSA, protein cocktail and dextran 500 kDa at 100 mg/mL. (FIG. 6B) Aggregation index values determined for blood, sodium chloride (NaCl) solution (i.e. saline), the protein cocktail, HSA, and dextran 500 kDa.

FIGS. 7A and 7B shows platelet aggregation tests on the control (saline, NaCl), HSA, Hextend, and the protein cocktail. (FIG. 7A) Collagen platelet aggregation. (FIG. 7B) ADP platelet aggregation. P<0.05 vs. no treatment. [symbol] vs. HSA. ^{§}P<0.05 vs. Hextend.

FIG. 8 illustrates some of the major roles of the protein components in the protein scavenging cocktail for treatment of various states of hemolysis. Proteins in the cocktail are highlighted in the green rectangles. Figure adapted with permission from Buehler and Karnaukhova(“WhenMight Transferrin, Hemopexin or Haptoglobin Administration Be of Benefit Following the Transfusion of Red Blood Cells?,” Buehler & Karnaukhova, 2018b).

FIG. 9 summarizes the effect on blood vessel diameter, blood velocity and blood flow of an example protein cocktail described herein in a model of hemorrhagic shock.

FIG. 10 summarizes the effect on functional capillary density (FCD) of an example protein cocktail described herein in a model of hemorrhagic shock.

FIG. 11 summarizes the effect on mean arterial pressure (MAP) and heart, rate (HR) of an example protein cocktail described herein in a model of hemorrhagic shock.

FIG. 12 summarizes the effect on blood parameters of an example protein cocktail described herein in a model of hemorrhagic shock.

FIG. 13 summarizes the effect on blood oxygen saturation levels of an example protein cocktail described herein in a model of hemorrhagic shock.

FIG. 14 illustrates the effect on blood oxygen saturation levels of an example protein cocktail in a model of hemorrhagic shock.

FIG. 15 illustrates the effect on blood oxygen saturation levels of a hydroxyethyl starch (HES) solution in a model of hemorrhagic shock.

FIG. 16 illustrates the effect on blood oxygen saturation levels of a blood control in a model of hemorrhagic shock.

FIG. 17 summarizes the effect of an example protein cocktail described herein in blood coagulation parameters.

FIG. 18 summarizes the effect of an example protein cocktail described herein in blood coagulation parameters.

FIG. 19 summarizes the effect of an example protein cocktail described herein in blood coagulation parameters.

FIG. 20 summarizes the effect of an example protein cocktail described herein in blood coagulation parameters.

FIGS. 21A-21F shows bilirubin and ferritin levels in blood and tissue from animals exchange transfused with mechanically hemolyzed blood plasma mixed with the protein cocktail (PC) or Dextran 70 kDa (Dex70). Sham indicates baseline levels in healthy animals. (FIG. 21A) Blood bilirubin, (FIG. 21B) blood ferritin, (FIG. 21C) splenic ferritin, (FIG. 21D) hepatic ferritin, (FIG. 21E) renal ferritin, and (FIG. 21F) cardiac ferritin. Markers measured from hepatic, splenic, renal, and cardiac tissue are normalized to the total protein content in the tissue. n = 5 animals/group. *p < 0.05; **p < 0.01, ***p < 0.001, ****p < 0.0001.

FIGS. 22A-22O shows Markers of renal, hepatic and cardiac tissue inflammation and injury from animals exchange transfused with mechanically hemolyzed blood plasma mixed with the protein cocktail (PC) or Dextran 70 kDa (Dex70). Sham indicated baseline levels in healthy animals. (FIG. 22A) hepatic aspartate aminotransferase (AST), (FIG. 22b) serum creatine, (FIG. 22C) blood urea nitrogen (BUN), (FIG. 22D) cardiac tumor necrosis factor alpha (TNF-α), (FIG. 22E) cardiac monocyte chemoattractant protein-1 (MCP-1), (FIG. 22F) hepatic alanine aminotransferase (ALT), (FIG. 22G) neutrophil gelatinase associated lipocalin (NGAL), (FIG. 22H) renal interleukin-1 (IL-1), (FIG. 22I) cardiac C-reactive protein (CRP), (FIG. 22J) cardiac atrial natriuretic peptide (ANP), (FIG. 22K) hepatic chemokine ligand 1 (CXCL1), (FIG. 22L) renal interleukin-6 (IL-6), (FIG. 22M) renal interleukin-10 (IL-10), (FIG. 22N) cardiac CXCL1, and (FIG. 22O) cardiac IL-6. Markers measured from renal and hepatic tissue (FIGS. 22D, 22E, 22H, 22I, 22J, 22K, 22L, 22M, 22N, and 22O) are normalized to the total protein content in the tissue, n = 5 animals/group. *p < 0.05; **p < 0.01, ***p < 0.001, ****p < 0.0001.

### DETAILED DESCRIPTION

The presently disclosed methods and the compositions as used in the disclosed methods seek to provide new therapies for the treatment of plasma protein imbalances or depletion. The plasma protein imbalance or depletion can be caused by, for example, hemorrhagic shock, burns, surgery, organ transplantation, a hypovolemic state, sepsis, trauma, subcutaneous trauma, kidney dialysis, traumatic brain injury, traumatic brain injury combined with hemorrhagic shock, or any combination thereof. In some embodiments, the plasma protein imbalance or depletion requires a plasma expander or a blood volume replacement. The protein compositions as used in the methods described herein provide an effective, inexpensive, and transportable fluid for treating such plasma protein imbalances or depletion.

The details of the disclosed methods and compositions can be understood more readily by reference to the following detailed description of specific aspects of the disclosed subject matter and the Examples and Figures included thereof.

Throughout this specification, various publications are referenced. The disclosures of these publications in their entireties are hereby incorporated by reference into this application in order to more fully describe the state of the art to which the disclosed subject matter pertains. The references disclosed are also individually and specifically incorporated by reference herein for the material contained in thein that is discussed in the sentence in which the reference is relied upon.

In this specification and in the claims that follow, reference will be made to a number of terms, which shall be defined to have the following meanings.

Throughout the description and claims of this specification the word “comprise” and other forms of the word, such as “comprising” and “comprises”, means including but not limited to, and is not intended to exclude, for example, other additives, components, integers, or steps.

As used in the description and the appended claims, the singular forms “a”, “an”, and “the” include plural referents unless the context clearly dictates otherwise.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint.

As used herein, by a “subject” is meant an individual. Thus, the “subject” can include domesticated animals (e.g., cats, dogs, etc.), livestock (e.g., cattle, horses, pig, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Administration” to a subject includes any route of introducing or delivery to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Systemic administration” refers to the introducing or delivery to a subject an agent via a route which introduces or delivers the agent to extensive areas of the subject’s body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically effective amount. For example, locally administered gents are easily detectable in the local vicinity of the point of administration but are undetectable or detectable at negligible amounts in distal parts of the subject’s body. Administration includes self-administration and the administration by another.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

The term “therapeutically effective” means that the amount of the composition used is of sufficient quantity to ameliorate one or more causes or symptoms of a disease or disorder. Such amelioration only requires a reduction or alteration, not necessarily elimination.

### Protein Compositions

Protein compositions isolated from plasma or a plasma fraction for use in the disclosed methods are provided that comprise:

from 5% by weight to 99% by weight (e.g., from 50% by weight to 95% by weight, from 5% by weight to 60% by weight, from 5% by weight to 25% by weight, from 5% by weight to 40% by weight, from 5% by weight to 30% by weight, or from 5% by weight to 25% by weight) haptoglobin, based on the total weight of all proteins in the composition; and from 1% by weight to 95% by weight (e.g., from 1% by weight to 40% by weight, from 5% by weight to 60% by weight, or from 10% by weight to 40% by weight) transferrin, based on the total weight of all proteins in the composition.

In some embodiments, the composition as used herein comprises:
from 5% to 15% by weight haptoglobin, based on the total weight of all proteins in the composition;
from 30% to 50% by weight albumin, based on the total weight of all proteins in the composition;
from 1% to 10% by weight hemopexin, based on the total weight of all proteins in the composition; and
from 30% to 40% by weight transferrin, based on the total weight of all proteins in the composition.

In some embodiments, the composition further comprises from 5% to 15% by weight vitamin-D binding protein, ceruloplasmin, or a combination thereof, based on the total weight of all proteins in the composition.

In some embodiments, the composition can be substantially free (i.e., the composition can include less than 0.5% by weight) of immunogenic proteins, such as antibodies.

In some embodiments, the composition is characterized by having residual hemoglobin as characterized by UV-visible spectroscopy of the Soret peak ranging from 402-407 nm. In some embodiments, the residual hemoglobin can be present in an amount less than 10% by weight (e.g., less than 5% by weight, less than 3% by weight, or less than 1% by weight), based on the total weight of all proteins in the composition.

### Haptoglobin (HP) and Transferrin (TF)

The protein compositions described herein may comprise from 5% by weight to 99% by weight, from 25% by weight to 99% by weight, from 30% by weight to 99% by weight, from 40% by weight to 99% by weight, from 50% by weight to 99% by weight, from 60% by weight to 99% by weight, from 95% by weight to 99% by weight, from 5% by weight to 95% by weight, from 25% by weight to 95% by weight, from 30% by weight to 95% by weight, from 40% by weight to 95% by weight, from 50% by weight to 95% by weight, from 60% by weight to 95% by weight, from 5% by weight to 60% by weight, from 25% by weight to 60% by weight, from 30% by weight to 60% by weight, from 40% by weight to 60% by weight, from 50% by weight to 60% by weight, from 5% by weight to 50% by weight, from 25% by weight to 50% by weight, from 30% by weight to 50% by weight, from 40% by weight to 50% by weight, from 5% by weight to 40% by weight, from 25% by weight to 40% by weight, from 30% by weight to 40% by weight, from 5% by weight to 30% by weight, from 25% by weight to 30% by weight, and from 5% to 25% by weight haptoglobin (Hp), based upon the total weight of all proteins in the composition.

Hp is an α-2 glycoprotein mainly responsible for scavenging cell-free Hb (Hb) (see Shih, A. W. Y., McFarlane, A. & Verhovsek, M. Haptoglobin testing in hemolysis: measurement and interpretation. Am. J. Hematol. 89, 443-7 (2014); and Yerbury, J. J., Kumita, J. R., Meehan, S., Dobson, C. M. & Wilson, M. R. α 2 - Macroglobulin and Haptoglobin Suppress Amyloid Formation by Interacting with Prefibrillar Protein Species. J. Biol. Chem. 284, 4246-4254 (2009)). The molecular weight (MW) of Hp varies from approximately 90-900 kDa due to its polymorphism (see Schaer, C. A. et al. Phenotype-specific recombinant haptoglobin polymers co-expressed with C1r-like protein as optimized hemoglobin-binding therapeutics. BMC Biotechnol. 18, 15 (2018); and Larsson, M., Cheng, T.-M., Chen, C.-Y. & J., S. Unique Assembly Structure of Human Haptoglobin Phenotypes 1-1, 2-1, and 2-2 and a Predominant Hp 1 Allele Hypothesis, in Acute Phase Proteins (InTech, 2013). doi:10.5772/56048). Although found in most bodily fluids of mammals, it is present in plasma at concentrations normally ranging from 0.5-3 mg/mL (see Muranjan, M., Nussenzweig, V. & Tomlinson, S. Characterization of the human serum trypanosome toxin, haptoglobin-related protein. J. Biol. Chem. 273, 3884-7 (1998); Yerbury, J. J., Rybchyn, M. S., Easterbrook-Smith, S. B., Henriques, C. & Wilson, M. R. The Acute Phase Protein Haptoglobin Is a Mammalian Extracellular Chaperone with an Action Similar to Clusterin. Biochemistry 44, 10914-10925 (2005); and Andersen, C. B. F. et al. Haptoglobin. Antioxid Redox Signal. 26, 814-831 (2017)). After binding to cell-free Hb, the Hb-Hp complex is scavenged by CD163+ macrophages and monocytes to clear the organism of toxic cell- free Hb (see Alayash, A. I., Andersen, C. B. F., Moestrup, S. K. & Bulow, L. Haptoglobin: the hemoglobin detoxifier in plasma. Trends Biotechnol. 31, 2-3 (2013)). When bound to Hp, the large size of the Hb-Hp complex prevents Hb extravasation into the tissue space, reducing NO scavenging and vasoconstriction (see Schaer, D. J., Buehler, P. W., Alayash, A. I., Belcher, J. D. & Vercellotti, G. M. Hemolysis and free hemoglobin revisited: exploring hemoglobin and hemin scavengers as a novel class of therapeutic proteins. Blood 121, 1276-84 (2013); Boretti, F. S. et al. Sequestration of extracellular hemoglobin within a haptoglobin complex decreases its hypertensive and oxidative effects in dogs and guinea pigs. J. Clin. Invest. 119, 2271-80 (2009); Belcher, J. D. et al. Haptoglobin and hemopexin inhibit vaso-occlusion and inflammation in murine sickle cell disease: Role of heme oxygenase-1 induction. PLoS One 13, e0196455 (2018); and Schaer, C. A. et al. Haptoglobin Preserves Vascular Nitric Oxide Signaling during Hemolysis. Am. J. Respir. Crit. Care Med. 193, 1111-22 (2016)). Furthermore, Hp binding to Hb prevents heme release from Hb, and lowers the ability of Hb to elicit oxidative damage and inflammation (see Belcher, J. D. et al. and Lim, S.-K., Ferraro, B., Moore, K. & Halliwell, B. Role of haptoglobin in free hemoglobin metabolism. Redox Rep. 6, 219-227 (2001)).

Other interesting properties of Hp are its intrinsic antioxidant potential, chaperone activity and binding to HMGB1 protein (See Schaer, C. A. et al.; Larsson, M. et al.; Tseng, C. F., Lin, C. C., Huang, H. Y., Liu, H. C. & Mao, S. J. T. Antioxidant role of human haptoglobin. Proteomics 4, 2221-2228 (2004); and Sultan, A., Raman, B., Rao, C. M. & Tangirala, R. The Extracellular Chaperone Haptoglobin Prevents Serum Fatty Acid-promoted Amyloid Fibril Formation of β Microglobulin, Resistance to Lysosomal Degradation, and Cytotoxicity. J. Biol. Chem. 288, 32326-32342 (2013)).

The protein compositions described herein may comprise from 1% by weight to 95% by weight, from 5% by weight to 95% by weight, from 10% by weight to 95% by weight, from 40% by weight to 95% by weight, from 60% by weight to 95% by weight, from 1% by weight to 60% by weight, from 5% by weight to 60% by weight, from 10% by weight to 60% by weight, from 40% by weight to 60% by weight, from 1% by weight to 40% by weight, from 5% by weight to 40% by weight, from 10% by weight to 40% by weight, from 1% by weight to 10% by weight, from 5% by weight to 10% by weight, or from 1% by weight to 5% by weight transferrin (Tf), based on the total weight of all proteins within the composition.

Tf is a ~80 kDa serum glycoprotein normally present at 2-4 mg/mL in the plasma. Each Tf molecule has two iron binding sites (binds to ferric iron, Fe³⁺) and the Tf-Fe complex maintains the iron in a non-reactive state. During states of hemolysis, Tf saturation with iron increases with a contaminant increase in non-transferrin bound iron. The increase in non-transferrin bound iron could lead to complications due to bacterial infection (see Hod, E. A. et al. Transfusion of human volunteers with older, stored red blood cells produces extravascular hemolysis and circulating non-transferrin-bound iron. Blood 118, 6675-6682 (2011)).

Tf may be a necessary co-therapeutic during intracerebral hemorrhage, since Hp and Hpx have been shown in vitro to increase iron-dependent neural cell damage when administered individually (see Chen-Roetling, J. & Regan, R. F. Haptoglobin Increases the Vulnerability of CD163- Expressing Neurons to Hemoglobin. J. Neurochem. 139, 586 (2016); and Chen-Roetling, J. Ma, S.-K., Cao, Y., Shah, A. & Regan, R. F. Hemopexin increases the neurotoxicity of hemoglobin when haptoglobin is absent. J. Neurochem. 145, 464-473 (2018)). Furthermore, addition of iron chelating agents decreased neural damage in vitro. However, both Hp and Hpx attenuate neural damage from intracerebral hemorrhage in vivo by both reducing oxidative damage from heme/Hb and stimulating HO-1 expression.

### Hemopexin (HPX)

In some embodiments, the composition can further comprise from 1% by weight to 75% by weight (e.g., from 2% by weight to 30% by weight, from 5% by weight to 20% by weight, or from 5% to 40% by weight) hemopexin (Hpx), based on the total weight of all proteins in the composition. For example, the composition can comprise from 1% by weight to 75% by weight, 2% by weight to 75% by weight, 5% by weight to 75% by weight, 20% by weight to 75% by weight, 30% by weight to 75% by weight, 40% by weight to 75% by weight, from 1% by weight to 40% by weight, 2% by weight to 40% by weight, 5% by weight to 40% by weight, 20% by weight to 40% by weight, 30% by weight to 40% by weight, from 1% by weight to 30% by weight, 2% by weight to 30% by weight, 5% by weight to 30% by weight, 20% by weight to 30% by weight, from 1% by weight to 20% by weight, 2% by weight to 20% by weight, 5% by weight to 20% by weight, from 1% by weight to 5% by weight, from 1% by weight to 5% by weight, or from 1% by weight to 2% by weight hemopexin, based on the total weight of all proteins in the composition.

Hpx is a ~60 kDa serum glycoprotein (~20% carbohydrate) with the highest affinity for free heme (K_{d} < 1 pM) (see Smith, A. Protection against Heme Toxicity: Hemopexin Rules, OK? in 311-338 (2013). doi:10.1142/9789814407755_0045; and Tolosano, E. & Altruda, F. Hemopexin: Structure, Function, and Regulation. DNA Cell Biol. 21, 297-306 (2002)). Each Hpx molecule can bind one heme molecule and its concentration in plasma ranges from 0.5-1.5 mg/mL (see Buehler, P. W. & Karnaukhova, E. When might transferrin, hemopexin or haptoglobin administration be of benefit following the transfusion of red blood cells? Curr. Opin. Hematol. 25, 452-458 (2018)). After binding to heme, the Hpx-heme complex has been shown to be cleared via two mechanisms: a high-affinity, low capacity (specific) system and a high-capacity low affinity system (selective) (see Smith, A. & Morgan, W. T. Hemopexin-mediated transport, of heme into isolated rat hepatocytes. J. Biol. Chem. 256, 10902-9 (1981)). The specific system is attributed to the CD91 receptor (low density lipoprotein receptor-related protein 1, LRP1) expressed on macrophages, hepatocytes, neurons, and syncytiotrophoblasts (see Buehler, P. W. & Karnaukhova, E. When might transferrin, hemopexin or haptoglobin administration be of benefit following the transfusion of red blood cells? Curr. Opin. Hematol. 25, 452-458 (2018); Smith, A.; Ascenzi, P. et al. Hemoglobin and heme scavenging. IUBMB Life (International Union Biochem. Mol. Biol. Life) 57, 749-759 (2005); and Smith, A. 71 Mechanisins of Cytoprotection by Hemopexin. in 217-355 (2011). doi:10.1142/9789814322386_0023). Unlike Hp, after intracellular heme release, Hpx is usually recycled and released intact back into circulation (see Smith, A.; Delanghe, J. R. & Langlois, M. R. Hemopexin: a review of biological aspects and the role in laboratory medicine. Clin. Chim. Acta 312, 13-23 (2001); and Smith, A. & Morgan, W. T. Haem transport to the liver by haemopexin. Receptor-mediated uptake with recycling of the protein. Biochem. J. 182, 47-54 (1979)). However, Hpx recycling has not been well defined and there is evidence that, during hemolytic states, serum Hpx levels decreases indicating that Hpx may be degraded upon receptor mediated uptake. The discrepancy in Hpx uptake and recycling has been attributed to the two different mechanisms of uptake (specific versus selective). At low heme levels, Hpx could be taken up and recycled by the specific LRP1/CD91 pathway, but at high heme levels, heme-Hpx is degraded to limit intracellular heme levels (cytoprotective). The studies that have demonstrated heme lysosomal degradation used high LRP1 expressing cells which could have overwhelmed the sorting nexin 17 mediated endosome pathway for recycling of LRP1, leading to Hpx degradation in the lysosome. Evidence for Hpx recycling comes from in vivo and in vitro studies in which gold-labeled Hpx was taken up into endosomes similar to holo-Tf recycling (see Smith, A. & Hunt, R. C. Hemopexin joins transferrin as representative members of a distinct class of receptor-mediated endocytic transport systems. Eur. J. Cell Biol. 53, 234-45 (1990)). Thus through receptor mediated heme transport, cells are protected from heme overload that can occur when excess free heme is present.

Similar to Hp binding to Hb, Hpx binding to heme prevents the oxidative reactions of heme from occurring (see Buehler, P. W. & Karnaukhova, E. When might transferrin, hemopexin or haptoglobin administration be of benefit following the transfusion of red blood cells? Curr. Opin. Hematol. 25, 452-458 (2018)). Furthermore, Hpx aids Hp in clearance of heme derived from cell-free Hb (see Smith, A. & McCulloh, R. J. Hemopexin and haptoglobin: allies against heme toxicity from hemoglobin not contenders. Front. Physiol. 6, 187 (2015)). Yet, contrary to the expected increase in heme uptake via Hpx, excess Hpx has been shown to reduce heme uptake into hepatic cells, likely as a mechanism to reduce intracellular iron toxicity (Latunde-Dada, G. O., Simpson, R. J. & McKie, A. T. Recent advances in mammalian haem transport. Trends in Biochemical Sciences 31, 182-188 (2006)). This mechanism may be of more importance in intracerebral hemolysis so as to reduce heme toxicity towards neural cells (see Chen-Roetling, J., Liu, W. & Regan, R. F. Hemopexin decreases hemin accumulation and catabolism by neural cells. Neurochem. Int. 60, 488-494 (2012)). In addition to heme scavenging originating from Hb, Hpx may be associated with heme clearance from cell-free Mb and from Hb-based red blood cell (RBC) substitutes.

Hpx also aids during hemolysis by inducing HO-1 and ferritin. These proteins protect the organism from the oxidative and inflammatory stress of heme during hemolysis. The importance of HO-1 has been evidenced as its gradual induction with repeated small heme doses has been shown to improve resistance against heme overload damage (see Vinchi, F., Gastaldi, S., Silengo, L., Altruda, F. & Tolosano, E. Hemopexin Prevents Endothelial Damage and Liver Congestion in a Mouse Model of Heme Overload. Am. J. Pathol. 173, 289-299 (2008)). Furthermore, HO-1 induction can induce lower wound scaring by reducing heme levels in the wound (A.D.T.G. Wagener, F. et al. The Heme-Heme Oxygenase System in Wound Healing; Implications for Scar Formation).

Recent evidence also indicates that the carbohydrate chains of native Hpx confers protection against heme oxidative reactions, which can limit the effectiveness of recombinant non- glycosylated Hpx.

### Human Serum Albumin (HSA)

In some embodiments, the composition can further include from 1% by weight to 70% by weight (e.g., from 5% by weight to 70% by weight, from 1% to 30% by weight, from 1% by weight to 15% by weight, or from 5% to 30% by weight) albumin (e.g., monomeric and polymeric albumin), based on the total weight of all proteins in the composition.

In some embodiments, the composition comprises from 1% by weight to 70% by weight, from 5% by weight to 70% by weight, from 15% by weight to 70% by weight, from 30% by weight to 70% by weight, from 1% by weight to 30% by weight, from 5% by weight to 30% by weight, from 15% by weight to 30% by weight, from 1% by weight to 15% by weight, from 5% by weight to 15% by weight, or from 1% by weight to 5% by weight albumin, based on the total weight of all proteins in the compositions.

Human serum albumin (HSA), is a 65 kDa non-glycosylated chain. Circulating at approximately 35-55 mg/mL in plasma, HSA is the most abundant plasma protein with a diverse set of functions. For example, HSA regulates oncotic pressure, binds and transports a variety of endogenous and exogenous molecules, and can have enzymatic and antioxidant properties (see Ascenzi, P., di Masi, A., Fanali, G. & Fasatio, M. Heme-based catalytic properties of human serum albumin. Cell death Discov. 1, 15025 (2015)). Clinical applications of albumin include, blood volume replacement, emergency shock treatment, treatment of burns and other cases of hypovolemia (see Taverna, M., Marie, A.-L., Mira, J.-P. & Guidet, B. Specific antioxidant properties of human serum albumin. Ann. Intensive Care 3, 4 (2013)). The benefits from HSA infusion have been largely attributed to its antioxidant properties. Its high concentration in plasma and free thiol group (Cys34 residue) accounts for 80% of thiols in plasma, making HSA the major extracellular source of free thiols. The Cys34 residue can scavenge various free radicals (HSA accounts for 70% of plasma free-radical trapping) involved in the damaging oxidative pathways of hemolysis such as hydrogen peroxide, peroxynitrite, and superoxide (see Buehler, P. W., D′Agnillo, F. & Schaer, D. J. Hemoglobin-based oxygen carriers: from mechanisms of toxicity and clearance to rational drug design. Trends Mol. Med. 16, 447- 457 (2010)).

Moreover, HSA can bind to both free heme and free iron (see Loban, A., Kime, R. & Powers, H. Iron-binding antioxidant potential of plasma albumin, Clin. Sci. (Lond). 93, 445-51 (1997)). Although at a lower affinity than Hpx (K_{d} ≈ 10 nM), heme binding to HSA decreases free heme-mediated oxidative damage. Furthermore, although at an equimolar heme concentration, HSA does not fully prevent lipid oxidation, at concentrations of 4× molar excess, HSA has been shown to prevent heme oxidative damage (see Miller, Y. I,, Folikman, Y. & Shaklai, N. The involvement of low-density lipoprotein in hemin transport potentiates peroxidative damage. Biochim. Biophys. Acta - Mol. Basis Dis. 1272, 119-127 (1995)). Yet, unlike Hpx, HSA itself is prone to oxidation due to the bound heme. Similarly, the iron binding properties of HSA prevents oxidative damage due to free iron. Furthermore, Hb mediated lipid peroxidation can be prevented via HSA administration. Thus, HSA can function as an iron and heme transport/carrier until Tf and Hpx can deliver them to their respective clearance receptors. Furthermore, kinetic studies have revealed that, upon heme release from Hb, the majority of heme is initially bound by low- and high-density lipoproteins (see Miller, Y. I. & Shaklai, N. Kinetics of hemin distribution in plasma reveals its role in lipoprotein oxidation. Biochim. Biophys. Acta 1454, 153-64 (1999)). Heme is then transferred first to albumin which then transports the heme to Hpx. Finally, the same mechanism of heme transport via albumin has been shown with respect to heme bound to the RBC membrane.

Another interesting property, of HSA related to hemolysis is its role in bilirubin transport. In addition to aiding in transport of catabolized heme, given the role of bilirubin as a physiological reductant, bilirubin-bound HSA can have enhanced antioxidant properties by preventing lipid peroxidation (see Neuzil, J. & Stocker, R. Free and albumin-bound bilirubin are efficient co-antioxidants for alpha-tocopherol, inhibiting plasma and low-density lipoprotein lipid peroxidation. J. Biol. Chem. 269, 16712-9 (1994)).

Interestingly conditions in which administration of HSA is clinically recommended may benefit from hemolysis treatment proteins. For example, during severe burns, septic shock, organ transplantation, or surgeries HSA can be administered as a plasma expander (see Liumbruno, G., Bennardello, F., Lattanzio, A., Piccoli, P. & Rossettias, G. Recommendations for the use of albumin and immunoglobulins. Blood Transƒus. 7, 216 (2009)). Yet, these conditions have also been shown to have hemolytic traits (see Effenberger-Neidnicht., K. & Hartmann, M. Mechanisms of Hemolysis During Sepsis. Inƒlammation 41, 1569-1581 (2018); Vermeulen Windsant, I. C. et al. Hemolysis during cardiac surgery is associated with increased intravascular nitric oxide consumption and perioperative kidney and intestinal tissue damage. Front. Physiol. 5, 340 (2014); Achkar, R., Chiba, A. K., Zampieri-Filho, J. P., Pestana, J. O. M. & Bordin, J. O. Hemolytic anemia after kidney transplantation: a prospective analysis. Transfusion 51, 2495-2499 (2011); Achkar, R., Chiba, A. K., Zampieri-Filho, J. P., Pestana, J. O. M. & Bordin, J. O. Hemolytic anemia after kidney transplantation: a prospective analysis. Transfusion 51, 2495-2499 (2011); Petz, L. D. Immune hemolysis associated with transplantation. Semin. Hematol. 42, 145-55 (2005); Norman, T. E. et al. Intravascular hemolysis associated with severe cutaneous burn injuries in five horses. J. Am. Vet. Med. Assoc. 226, 2039-43, 2002 (2005); and Endoh, Y., Kawakami, M., Orringer, E. P., Peterson, H. D. & Meyer, A. A. Causes and time course of acute hemolysis after burn injury in the rat. J. Burn Care Rehabil. 13, 203-9). Thus, administration of a protein cocktail containing both HSA and hemolysis scavenging proteins could yield better patient outcomes.

Studies have demonstrated a receptor for heme which can explain heme delivery via heme- albumin (see Taketani, S. Aquisition, Mobilization and Utilization of Cellular Iron and Heme: Endless Findings and Growing Evidence of Tight Regulation. Tohoku J. Exp. Med. 205, 297-318 (2005); Worthington, M. T., Cohn, S. M., Miller, S. K., Luo, R. Q. & Berg, C. L. Characterization of a human plasma membrane heme transporter in intestinal and hepatocyte cell lines. Am. J. Physiol. Liver Physiol. 280, G1172-G1177 (2001), Noyer, C. M., Immenschuh, S., Liem, H. H., Muller-Eberhard, U. & Wolkoff, A. W. Initial heme uptake from albumin by short-term cultured rat hepatocytes is mediated by a transport mechanism differing from that of other organic anions. Hepatology 28, 150-155 (1998); Taketani, S. et al. Hemopexin from four species inhibits the association of heme with cultured hepatoma cells or primary rat hepatocytes exhibiting a small number of species specific hemopexin receptors. Hepatology 27, 808-814 (1998); and Korolnek, T. & Hamza, I. Like iron in the blood of the people: the requirement for heme trafficking in iron metabolism. Front. Pharmacol. 5, 126 (2014)). Thus direct metabolism of heme can lead to its increased appearance in the bile, which can make the bile appear black in color (see Siegert, S. W. K. & Holt, R. J. Physicochemical properties, pharmacokinetics, and pharmacodynamics of intravenous hematin: a literature review. Adv. Ther. 25, 842-857 (2008)). Furthermore, both heme and heme-albumin have been shown to have similar HO-1 expression profiles with both leading to higher HO-1 levels than heme-Hpx (see Tolosano, E., Fagoonee, S., Morello, N., Vinchi, F. & Fiorito, V. Heme Scavenging and the Other Facets of Hemopexin. Antioxid. Redox Signal. 12, 305-320 (2010)). HSA has also been shown to reduce neural heme toxicity at equimolar concentrations, although to a lower extent that Hpx. Further evidence for the non-crucial role of Hpx in heme transport was shown in Hpx knockout which did not show differences in heme catabolism compared to wild type (see Tolosano, E. et al. Defective recovery and severe renal damage after acute hemolysis in hemopexin-deficient mice. Blood 94, 3906-14 (1999)). This was not the case for Hp knockout mice, which had higher susceptibility to hemolysis, indicating Hpx as a second line of defense (see Lim, S. K. et al. Increased susceptibility in Hp knockout mice during acute hemolysis. Blood 92, 1870–7 (1998). This is further evidenced by experiments demonstrating that, in general, plasma Hpx levels only decrease upon decrease in Hp levels (see Muller-Eberhard, U., Javid, J., Liem, H. H., Hanstein, A. & Hanna, M. Plasma concentrations of hemopexin, haptoglobin and heme in patients with various hemolytic diseases. Blood 32, 811-5 (1968)). Yet, there is evidence that Hpx may play an active role in heme capture even at normal Hp levels (see Smith, A. & McCulloh, R. J. Hemopexin and haptoglobin: allies against heme toxicity from hemoglobin not contenders. Front. Physiol. 6, (2015)). Furthermore, Hpx knockout mice did show higher renal damage and lipid peroxidation, likely due to the lack of heme capture from Hb by Hpx leading to higher Hb levels in the Hpx knockout mice. Furthermore, lack of Hpx led to an increase in Hp transcription compared to wild type. Thus, Hpx is considered to have a primary function to reduce heme toxicity and not have a major role in iron metabolism.

Another benefit of HSA as a component in the scavenging protein cocktail is its extensive ligand binding properties (see Fasano, M. et al. The extraordinary ligand binding properties of human serum albumin. IUBMB Life 57, 787-796 (2005)). This allows for a flexible delivery vehicle of drugs for treatment of the desired condition. For example, as the major store of NO in vivo, HSA in the protein mixture may be used to deliver NO to the vasculature during states of hemolysis, thus preventing hypertension (see Stamler, J. S. et al. Nitric oxide circulates in mammalian plasma primarily as an S-nitroso adduct of serum albumin. Proc. Natl. Acad. Sci. 89, 7674-7677 (1992); Rungatscher, A. et al. S-nitroso human serum albumin attenuates pulmonary hypertension, improves right ventricular-arterial coupling, and reduces oxidative stress in a chronic right ventricle volume overload model. J. Hear. Lung Transplant. 34, 479-488 (2015); and Orie, N. N., Vallance, P., Jones, D. P. & Moore, K. P. S -nitroso-albumin carries a thiol- labile pool of nitric oxide, which causes venodilation in the rat. Am. J. Physiol. Circ. Physiol. 289, H916-H923 (2005)). Nitrite infusions have already been shown to restrict Hb hypertension during hemolysis (see Minneci, P. C. et al. Nitrite reductase activity of hemoglobin as a systemic nitric oxide generator mechanism to detoxify plasma hemoglobin produced during hemolysis. Am. J. Physiol. Circ. Physiol. 295, H743-H754 (2008)). In one strategy, NO delivery would require binding of NO to the free Cys34 of HSA to form S-NO HSA (HSA-SNO) prior to administration of the cocktail, but could serve as a means to increase NO levels in the blood that may have been scavenged due to cell-free Hb. HSA-SNO may also be used in wound healing applications while the scavenging proteins would prevent wound infections via iron sequestration (see Ganzarolli de Oliveira, M. S-Nitrosothiols as Platforms for Topical Nitric Oxide Delivery. Basic Clin. Pharmacol. Toxicol. 119, 49-56 (2016); and LUO, J. & CHEN, A. F. Nitric oxide: a newly discovered function on wound healing. Acta Pharmacol. Sin. 26, 259-264 (2005)). Finally, HSA-SNO can also have application in the treatment of cyanide poisoning (see Leavesley, H. B., Li, L., Mukhopadhyay, S., Borowitz, J. L. & Isom, G. E. Nitrite-Mediated Antagonism of Cyanide Inhibition of Cytochrome c Oxidase in Dopainine Neurons. Toxicol. Sci. 115, 569-576 (2010)).

### GC Globulin/Vitamin-D Binding Protein (VDB) and Ceruloplasmin

In some embodiments, the composition can further include from 5% by weight to 15% by weight vitamin-D binding protein, ceruloplasmin, or a combination thereof, based on the total weight of all proteins in the composition.

Gc globulin, also known as vitamin-D binding protein (VDB) is a back-up actin scavenging protein (see Chun, R. F. New perspectives on the vitamin D binding protein. Cell Biochem. Funct. 30, 445-456 (2012); and Meier, U., Gressner, O., Lammert, F. & Gressner, A. M. Gc-Globulin: Roles in Response to Injury. Clin. Chem. 52, 1247-1253 (2006)). Serum actin is also a toxic species and hemolysis has been shown to increase actin levels, which saturate the binding capacity of the natural actin scavenger gelsolin (see Piktel, E., Levental, I., Duma’s, B., Janmey, P. A. & Bucki, R. Plasma Gelsolin: Indicator of Inflammation and Its Potential as a Diagnostic Tool and Therapeutic Target. lnt. J. Mol. Sci. 19, (2018); Peddada, N., Sagar, A. & Garg, R. Plasma gelsolin: A general prognostic marker of health. Med. Hypotheses 78, 203-210 (2012); and Smith, D., Janmey, P., Sherwood, J., Howard, R. & Lind, S. Decreased plasma gelsolin levels in patients with Plasmodium falciparum malaria: a consequence of hemolysis? Blood 72, (1988)).

Ceruloplasmin (Cp) is a ~120 kDa serum protein responsible for binding and transport of copper (see Hellman, N. E. & Gitlin, J. D. CERULOPLASMIN METABOLISM AND FUNCTION. Annu. Rev. Nutr. 22, 439-458 (2002)). Furthermore, Cp has a major role in iron metabolism as a ferroxidase for oxidation of Fe²⁺ into Fe³⁺ and for stabilization of ferroportin (cellular iron exporter) (see Ramos, D. et al. Mechanism of Copper Uptake from Blood Plasma Ceruloplasmin by Mammalian Cells. PLoS One 11, e0149516 (2016); and De Domenico, I. et al. Ferroxidase activity is required for the stability of cell surface ferroportin in cells expressing GPI-ceruloplasmin. EMBO J. 26, 2823-31 (2007)). Oxidation of iron to Fe³⁺ is required for iron binding to transferrin (transport) or ferritin (storage) (see de Silva, D. & Aust, S. D. Stoichiometry of Fe(II) oxidation during ceruloplasmin-catalyzed loading of ferritin. Arch. Biochem. Biophys. 298, 259-264 (1992); Samokyszyns, V. M., Miller, D. M., Reif, D. W. & Austq, S. D. Inhibition of Superoxide and Ferritin-dependent Lipid Peroxidation by Ceruloplasmin*. OF BIOLOGICAL CHEMISTRY 264, (1989); and Silva, D. M. de & Aust, S. D. Ferritin and ceruloplasmin in oxidative damage: review and recent findings. Can. J. Physiol. Pharmacol. 71, 715-720 (1993)). Thus, Cp is vital for proper iron metabolism and genetic Cp deficiencies lead to accumulation of iron in organs (see Dubick, M., Barr, J., Keen, C. & Atkins, J. Ceruloplasmin and Hypoferremia: Studies in Burn and Non-Burn Trauma Patients. Antioxidants 4, 153-169 (2015)). Moreover, low Cp levels have been associated with patients having a high risk for acute organ failure (see Dauberschmidt, R. et al. Changes in ceruloplasmin activity and lactate concentration in patients at high risk of acute organ system failure. Clin. Chim. Acta. 199, 167-72 (1991)). It has also been shown that Cp activity decreases during trauma and burn injury which contributes to inflammation and hypoferremia. Severe burns have also been associated with low Cp levels (see Cunningham, J. J., Lydon, M. K., Emerson, R. & Harmatz, P. R. Low ceruloplasmin levels during recovery from major burn injury: Influence of open wound size and copper supplementation. Nutrition 12, 83-88 (1996)).

Studies have also demonstrated that copper may be co-endocytosed with heme-Hpx given that copper binding to Hpx intracellularly may aid in heme release. Thus, copper transport may play an important role for controlled transport and metabolism of heme.

### Preparation of Protein Compositions

The protein compositions as used in the methods described herein can be isolated from plasma or a fraction thereof. In some embodiments, isolating the protein composition from plasma or a fraction thereof can comprise the steps of (i) clarifying the plasma or traction thereof; (ii) filtering the clarified plasma or a fraction thereof by ultrafiltration against a filtration membrane, thereby forming a retentate fraction comprising of proteins having a molecular weight of greater than about 100 kDa and a permeate fraction comprising the serum proteins described herein having a molecular weight of less than about 100 kDa, and (iii) concentrating or further purifying the permeate fraction, thereby forming a second retentate fraction comprising a blend of proteins having a molecular weight below about 100 kDa and above a cutoff value and a second permeate fraction comprising serum proteins and other impurities having a molecular weight below the cutoff value, wherein the blend of proteins in the second retentate fraction comprises low molecular weight haptoglobin, transferrin, hemopexin, albumin, or a combination thereof. The cutoff value can be from about 20 kDa to about 70 kDa, such as from about 30 kDa to about 60 kDa

The plasma or fraction thereof can comprise plasma fraction IV, plasma fraction V, a fraction of precipitated plasma (from salting out, polyethylene glycol, zinc chloride, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration.

In these methods, the second retentate fraction can include a blend of proteins (e.g., low molecular weight haptoglobin, transferrin, hemopexin, albumin, or a combination thereof) that can bind and detoxify cell-free hemoglobin, free iron, and/or free heme. Accordingly, the second retentate fraction can be administered to a subject in need thereof, for example, to treat hemorrhagic shock a described herein. In some examples, the second retentate fraction can be administered prophylactically to a subject to prevent damage associated with anticipated hemorrhagic shock.

In other embodiments, methods for the compositions as used herein can comprise (i) filtering the plasma or fraction thereof by ultrafiltration against a first filtration membrane, thereby forming a first retentate fraction comprising serum proteins having a molecular weight above a first cutoff value and a first permeate fraction comprising most of the haptoglobin and serum proteins having a molecular weight below the first cutoff value; and (ii) filtering the first permeate fraction by ultrafiltration against a second filtration membrane, thereby forming a second retentate fraction comprising small amounts of Hp2-1, Hp2-2, and serum proteins having a molecular weight below the first cutoff value and above a second cutoff value; and a second permeate fraction comprising Hp2-1, Hp2-2, and serum proteins having a molecular weight below the second cutoff value. In some cases, the method can further comprise (iii) filtering the second permeate fraction by tangential-flow filtration against a third filtration membrane, thereby forming a third retentate fraction comprising Hp2-1 and Hp2-2 having a molecular weight below the second cutoff value and above a third cutoff value; and a third permeate fraction comprising low molecular weight haptoglobin, serum proteins and other impurities having a molecular weight below the third cutoff value. In some cases, the method can further comprise (iv) filtering the third permeate fraction comprising low molecular weight haptoglobin, serum proteins and other impurities by ultrafiltration against a fourth filtration membrane, thereby forming a fourth retentate fraction comprising a blend of proteins having a molecular weight below the third cutoff value and above a fourth cutoff value and a fourth permeate fraction comprising of serum proteins and other impurities having a molecular weight below the fourth cutoff value, wherein the blend of proteins in the fourth retentate fraction comprises low molecular weight haptoglobin, transferrin, hemopexin, albumin, or a combination thereof.

The first cutoff value can be from about 650 kDa to about 1000 kDa. The second cutoff value can be from about 300 kDa to about 700 kDa. The third cutoff value can be from about 70 kDa to about 200 kDa. The fourth cutoff value can be from about 20 kDa to about 70 kDa. In certain examples, the first cutoff value can be about 750 kDa, the second cutoff value can be about 500 kDa, and the third cutoff value can be about 100 kDa. The fourth cutoff value can be about 30 kDa or about 50 kDa.

The plasma or fraction thereof can comprise plasma fraction IV, plasma fraction V, a fraction of precipitated plasma (from salting out, or equivalent) or a combination thereof.

Clarifying the plasma or a fraction thereof can comprise removing suspended solids from the plasma or fraction thereof. Removing suspended solids from the plasma or fraction thereof can comprise filtering (via ultrafiltration, microfiltration, depth filtration or equivalent) the plasma or a fraction thereof, contacting the plasma or a fraction thereof with a salting out agent (e.g., ammonium sulfate), an adsorbing agent (e.g., ethacridine lactate), or a combination thereof. Further clarification may be implemented through addition of a lipid-binding agent such as fumed silica (such as fumed silica sold under the tradename Aerosil 380®, or similar), clay, bentonite, terra alba, active carbon, or a combination thereof.

In some embodiments, the ultrafiltration can comprise tangential-flow filtration.

In these methods, the fourth retentate fraction can include a blend of proteins (e.g., low molecular weight haptoglobin, transferrin, hemopexin, albumin, or a combination thereof) that can bind and detoxify free hemoglobin, free iron, and/or free heme. Accordingly, the fourth retentate fraction can be administered to a subject in need thereof, for example, to treat hemorrhagic shock. In some examples, the fourth retentate fraction can be administered prophylactically to a subject to prevent damage associated with anticipated hemorrhagic shock.

### Methods for Treating Plasma Protein Imbalances or Depletion

Provided herein are methods for treating or preventing plasma protein imbalances or depletion using protein compositions as described herein. The plasma protein imbalance or depletion can be caused by, for example, hemorrhagic shock, burns, surgery, organ transplantation, a hypovolemic state, a hypervolemic state, sepsis, trauma, subcutaneous trauma, kidney dialysis, traumatic brain injury, traumatic brain injury combined with hemorrhagic shock, a coagulation disorder, or any combination thereof. In some embodiments, the plasma protein imbalance or depletion requires a plasma expander, a blood volume replacement or an extracorporeal pump priming fluid.

In some embodiments, the method comprises administering to the subject a therapeutically effective amount of a protein composition comprising:

from 5% by weight to 99% by weight (e.g., from 50% by weight to 95% by weight, from 5% by weight to 60% by weight, from 5% by weight to 25% by weight, from 5% by weight to 40% by weight, from 5% by weight to 30% by weight, or from 5% by weight to 25% by weight) haptoglobin, based on the total weight of all proteins in the composition; and from 1% by weight to 95% by weight (e.g., from 1% by weight to 40% by weight, from 5% by weight to 60% by weight, or from 10% by weight to 40% by weight) transferrin, based on the total weight of all proteins in the composition.

In some embodiments, a method is provided for treating or preventing plasma protein imbalances or depletion (e.g., plasma protein imbalances or depletion caused by hemorrhagic shock) in a subject in need thereof, comprising administering to the subject a therapeutically effective amount of a protein composition comprising:
from 5% to 15% by weight haptoglobin, based on the total weight of all proteins in the composition;
from 30% to 50% by weight albumin, based on the total weight of all proteins in the composition;
from 1% to 10% by weight hemopexin, based on the total weight of all proteins in the composition; and
from 30% to 40% by weight transferrin, based on the total weight of all proteins in the composition.

In some embodiments, the composition as used in the method described herein further comprises from 5% to 15% by weight vitamin-D binding protein, ceruloplasmin, or a combination thereof, based on the total weight of all proteins in the composition.

In certain embodiments, the plasma protein imbalance or depletion can be caused by hemorrhagic shock. Hemorrhagic shock is subset of hypovolemic shock resulting from blood loss. Traumatic injury is by far the most common cause of hemorrhagic shock, particularly blunt and penetrating trauma, followed by upper and lower gastrointestinal sources, such as gastrointestinal (GI) bleed. Other causes include bleed from an ectopic pregnancy, bleeding from surgical intervention, or vaginal bleed. Obstetrical, vascular, iatrogenic, and even urological sources have all been described. Bleeding may be either external or internal. A substantial amount of blood loss to the point of hemodynamic compromise may occur in the chest, abdomen, or retroperitoneum. The thigh itself can hold up to one to two liters of blood. Localizing and controlling the source of bleeding is of utmost importance to the treatment of hemorrhagic shock.

The most-commonly-seen causes that lead to hemorrhagic shock in order of frequency include: blunt or penetrating trauma including multiple fracture absent from vessel impairment; upper gastrointestinal bleeding (such as variceal hemorrhage or peptic ulcer) or lower gastrointestinal bleeding (such as diverticular), and arteriovenous malformation. Less common causes include intra-operative and post-operative bleeding, abnormal aortic rupture or left ventricle aneurysm rupture, aortic-enteric fistula, hemorrhagic pancreatitis, iatrogenic (such as inadvertent biopsy of an arteriovenous malformation), severed artery, tumor of abscess erosion into major vessels, post-partum hemorrhage, uterine or vaginal hemorrhage owing to infection, tumors, lacerations, spontaneous peritoneal hemorrhage caused by bleeding diathesis, and ruptured hematoma.

Hemorrhagic shock is due to the depletion of intravascular volume through blood loss to the point of being unable to match the tissues demand for oxygen. As a result, mitochondria are no longer able to sustain aerobic metabolism and switch to the less efficient anaerobic metabolism to meet the cellular demand for adenosine triphosphate. In the latter process, pyruvate is provided and converted to lactic acid to regenerate nicotinamide adenine dinucleotide (NAD+) to maintain some degree of cellular respiration in the absence of oxygen.

The body compensates for volume loss by increasing heart rate and contractility, followed by baroreceptor activation resulting in sympathetic nervous system activation and peripheral vasoconstriction. Typically, there is a slight increase in diastolic blood pressure with narrowing of the pulse pressure. As diastolic ventricular filling continues to decline and cardiac output decreases, systolic blood pressure drops.

Due to sympathetic nervous system activation, blood is diverted away from the noncritical organs and tissues to preserve blood supply to vital organs such as the heart and brain. While prolonging heart and brain function, this also leads to other tissues being further deprived of oxygen causing more lactic acid production and worsening acidosis. The worsening acidosis and hypoxemia, if left uncorrected, eventually causes the loss of peripheral vasoconstriction, worsening hemodynamic compromise, and death.

The body’s compensation varies by cardiopulmonary comorbidities, age, and vasoactive medications. Due to these factors, heart rate and blood pressure responses are extremely variable and therefore cannot be relied upon as the sole means of diagnosis.

A key factor in the pathophysiology of hemorrhagic shock is the development of trauma-induced coagulopathy. Coagulopathy develops as a combination of several processes. The simultaneous loss of coagulation factors via hemorrhage, hemodilution with resuscitation fluids, and coagulation cascade dysfunction secondary to acidosis and hypothermia have been traditionally thought to be the cause of coagulopathy in trauma. However, this traditional model of trauma-induced coagulopathy may be too limited. Further studies have shown that a degree of coagulopathy begins in 25-56% of patients before initiation of the resuscitation. This has led to the recognition of trauma-induced coagulopathy as the sum of two distinct processes: acute coagulopathy of trauma and resuscitation-induced coagulopathy.

Trauma-induced coagulopathy is acutely worsened in the presence of acidosis and hypothermia. The activity of coagulation factors, fibrinogen depletion, and platelet quantity are all adversely affected by acidosis. Hypothermia (less than 34° C.) compounds coagulopathy by impairing coagulation and is an independent risk factor for death in hemorrhagic shock.

The shock index (SI) is clinically employed to determine the scope or emergence of shock, defined as the ratio of heart rate/systolic blood pressure. An Sl greater than 0.6 is defined as clinical shock. The SI correlates with the extent of hypovolemia and thus may facilitate the early identification of severely injured patients threatened by complications due to blood loss and therefore needing urgent, treatment. Patients are classified by the shock index as belonging to group I (SI <0.6, no shock), group II (0.6<SI<1.0, mild shock), group III (1.0<SI<1.4, moderate shock), and group IV (SI>1.4, severe shock).

Further, recognizing the degree of blood loss via vital sign and mental status abnormalities are important. The American College of Surgeons Advanced Trauma Life Support (ATLS) hemorrhagic shock classification links the amount of blood loss to expected physiologic responses in a healthy 70 kg patient. Total circulating blood volume accounts for approximately 7% of total body weight and equals approximately five liters in the average 70 kg male patient.

Class 1: volume loss up to 15% of total blood volume (approximately 750 mL). Heart rate is minimally elevated or normal. Typically, there is no change in blood pressure, pulse pressure, or respiratory rate.

Class 2: volume loss from 15% to 30% of total blood volume (from 750 to 1500 mL). Heart rate and respiratory rate become elevated (100 BPM to 120 BPM, 20 RR to 24 RR). Pulse pressure begins to narrow, but systolic blood pressure may be unchanged to slightly decreased.

Class 3: volume loss from 30% to 40% of total blood volume (from 1500 to 2000 mL). A significant drop in blood pressure and changes in mental status occur. Heart rate and respiratory rate are significantly elevated (more than 120 BPM). Urine output declines. Capillary refill is delayed.

Class 4: volume loss over 40% of total blood volume. Hypotension with narrow pulse pressure (less than 25 mmHg). Tachycardia becomes more pronounced (more than 120 BPM), and mental status becomes increasingly altered. Urine output is minimal or absent. Capillary refill is delayed.

In the setting of trauma, an algorithmic approach via the primary and secondary surveys are suggested by ATLS. Physical exam and radiological evaluations can help localize sources of bleeding. A trauma ultrasound, or Focused Assessment with Sonography for Trauma (FAST), has been incorporated in many circumstances into the initial surveys. The specificity of a FAST scan has been reported above 99%, but a negative ultrasound does not rule out intra-abdominal pathology.

With a broader understanding of the pathophysiology of hemorrhagic shock, treatment in trauma has expanded from a simple massive transfusion method to a more comprehensive management strategy of “damage control resuscitation”. The concept of damage control resuscitation focuses on permissive hypotension, hemostatic resuscitation, and hemorrhage control to adequately treat the “lethal triad” of coagulopathy, acidosis, and hypothermia that occurs in trauma.

Hypotensive resuscitation has been suggested for the hemorrhagic shock patient without head trauma. The aim is to achieve a systolic blood pressure of 90 mmHg in order to maintain tissue perfusion without inducing re-bleeding from recently clotted vessels. Permissive hypotension is a means of restricting fluid administration until hemorrhage is controlled while accepting a short period of suboptimal end-organic perfusion. Studies regarding permissive hypotension have yielded conflicting results and must take into account type of injury (penetrative versus blunt), the likelihood of intracranial injury, the severity of the injury, as well as proximity to a trauma center and definitive hemorrhage control.

The quantity, type of fluids to be used, and endpoints of resuscitation remain topics of much study and debate. For crystalloid resuscitation, normal saline and lactated ringers are the most commonly used fluids. Normal saline has the drawback of causing a non-anion gap hyperchloremic metabolic acidosis due to the high chloride content, while lactated ringers can cause a metabolic acidosis as lactate metabolism regenerates into bicarbonate.

Recent trends in damage control resuscitation focus on “hemostatic resuscitation” which pushes for early use of blood products rather than an abundance of crystalloids in order to minimize the metabolic derangement, resuscitation-induced coagulopathy, and the hemodilution that occurs with crystalloid resuscitation. The end goal of resuscitation and the ratios of blood products remain at the center of much study and debate. A recent study has shown no significant difference in mortality at 24 hours or 30 days between ratios of 1:1:1 and 1:1:2 of plasma to platelets to packed RBCs. However, patients that receive the more balance ratio of 1:1:1 were less likely to die as a result of exsanguination in 24 hours and were more likely to achieve hemostasis. Additionally, reduction in time to first plasma transfusion has shown a significant reduction in mortality in damage control resuscitation.

In addition to blood products, products that prevent the breakdown of fibrin in clots, or antifibrinolytics, have been studied for their utility in the treatment of hemorrhagic shock in the trauma patient. Several antifibrinolytics have been shown to be safe and effective in elective surgery. The CRASH-2 study was a randomized control trial of tranexamic acid versus placebo in trauma that was shown to decrease overall mortality when given in the first eight hours of injury. Follow-iip analysis shows additional benefit of tranexamic acid when given in the first three hours after surgery.

Damage control resuscitation is to occur with prompt intervention to control the source of bleeding. Strategies may differ depending on proximity to definitive treatment.

For patients in hemorrhagic shock, early use of blood products over crystalloid resuscitation results in better outcomes. Balanced transfusion using 1:1: 1 or 1:1:2 of plasma to platelets to packed red blood cells results in better homeostasis. Anti-fibrinolytic administration to patients with severe bleeding within 3 hours of traumatic injury appears to decrease death from major bleeding. Research on oxygen-carrying substitutes as an alternative to packed red blood cells is ongoing, although no blood substitutes have been approved for use in the United States.

Trauma remains a leading cause of death worldwide with approximately half of these attributed to hemorrhage. In the United States in 2001, trauma was the third leading cause of death overall, and the leading cause of death in those aged 1 to 44 years. While trauma spans all demographics, it disproportionately affects the young with 40% of injuries occurring in ages 20 to 39 years. Of this 40%, the greatest incidence was in the 20 to 24-year-old range.

The preponderance of hemorrhagic shock cases resulting from trauma is high. During one year, one trauma center reported 62.2% of massive transfusions occur in the setting of trauma. The remaining cases are divided among cardiovascular surgery, critical care, cardiology, obstetrics, and general surgery, with trauma utilizing over 75% of the blood products.

As patients age, physiological reserves decrease, the likelihood of anticoagulant use increase, and the number of comorbidities increases. Due to this, elderly patients are less likely to handle the physiological stresses of hemorrhagic shock and may decompensate more quickly.

The compositions as used in the methods described herein can be administered by any suitable method and technique presently or prospectively known to those skilled in the art. For example, the active components described herein can be formulated in a physiologically- or pharmaceutically-acceptable form and administered by any suitable route known in the art including, for example, parenteral routes of administering. As used herein, the term “parenteral” includes subcutaneous, intradermal, intravenous, intramuscular, intraperitoneal, and intrasternal administration, such as by injection. Administration of the disclosed composition can be a single administration, or at continuous and distinct intervals as can be readily determined by a person skilled in the art.

The protein compositions may be administered in such amounts, time, and route deemed necessary in order to achieve the desired result. The exact amount of the protein composition will vary from subject to subject, depending on the species, age, and general condition of the subject, the severity of hemorrhagic shock, the particular active ingredient, its mode of administration, its mode of activity, and the like. The protein composition is preferably formulated in dosage unit form for ease of administration and uniformity of dosage. It will be understood, however, that the total daily usage of the composition will be decided by the attending physician within the scope of sound medical judgment. The specific therapeutically effective dose level for any particular subject will depend upon a variety of factors including the severity of hemorrhagic shock; the specific composition employed; the age, body weight, general health, sex and diet of the patient; the time of administration, and route of administration, the duration of the treatment, drugs used in combination or coincidental with the specific composition employed; and like factors well known in the medical arts.

The exact amount of the composition required to achieve a therapeutically or prophylactically effective amount will vary from subject to subject, depending on species, age, and general condition of a subject, severity of the side effects or disorder, identity of the particular protein components of the composition, mode of administration, and the like. The amount to be administered to, for example, a child or an adolescent can be determined by a medical practitioner or person skilled in the art and can be lower or the same as that admini stered to an adult.

Useful dosages of the compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder (i.e., hemorrhagic shock) are affected. The dosage should not be so large as to cause adverse side effects, such as unwanted cross-reactions, anaphylactic reactions, and the like. Generally, the dosage will vary with the age, condition, sex and extent of the disorder in the patient and can be determined by one of skill in the art. The dosage can be adjusted by the individual physician in the event of any counterindications. Dosage can vary, and can be administered in one or more dose administrations daily, for one or several days.

### EXAMPLES

By way of non-limiting illustration, examples of certain embodiments of the present disclosure are given below.

### Example 1. Purification and Analysis of a Protein Cocktail Capable of Scavenging Cell-Free Hemoglobin, Heme and Iron

### Materials and Methods

Materials. Sodium phosphate dibasic, sodium phosphate monobasic, sodium chloride, and fumed silica (S5130) were purchased (Sigma Aldrich, St. Louis, MO), and 0.2 µm polyethersulfone syringe filters were also purchased (Merck Millipore, Bellerica, MA). Protein separation was performed on a TFF system (KrosFlo® Research II, Repligen, Waltham, MA). The TFF system was equipped with various HF filter modules (Repligen, Waltham, MA). FIV paste from the modified Cohn process of Kistler and Nitschmann was purchased (Seraplex, Inc., Pasadena, CA).

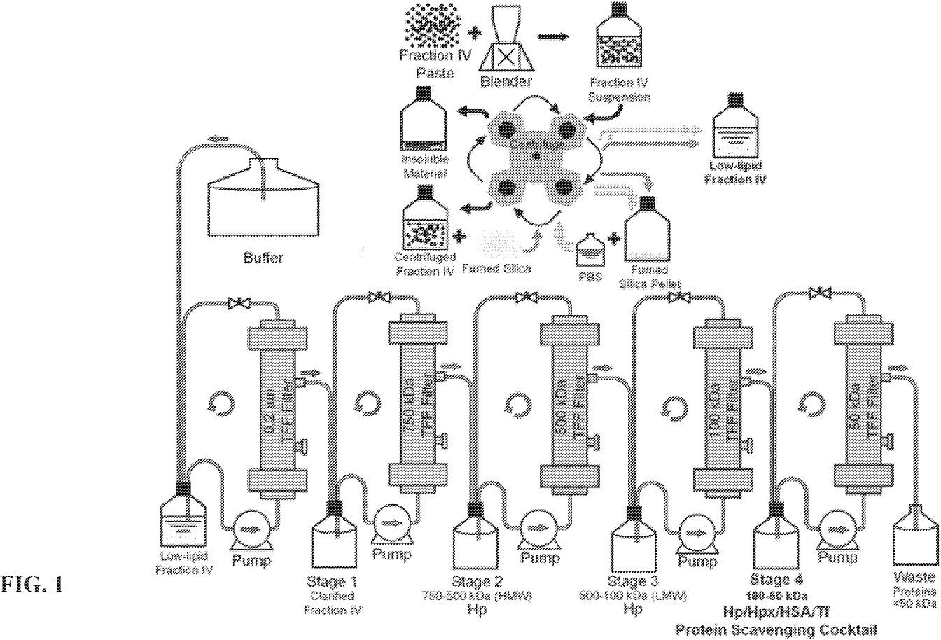

Hb, Heme and Iron Scavenging Protein Cocktail ^{_}Purification via TFF, Purification of the protein cocktail followed the TFF Hp purification method with the addition of one additional TFF stage at the end of the original process (Pires, I. S. & Palmer, A. F. Biotechnol. Prog. 2020, 36, e3010). Briefly, 500 g of FIV was suspended in 5 L of phosphate buffered saline (PBS) and homogenized in a blender. After stirring overnight at 4° C., the ∼ 5 L solution was centrifuged to remove undissolved lipids. Fumed silica was then added to the supernatant, left to stir overnight at 4° C., and then re-centrifuged with two PBS washes of the fumed silica pellet. The resulting protein solution was then clarified on a 0.2 µm HF filter and then bracketed using a series of HF modules with decreasing MW cutoff (MWCO) (750, 500, and 100 kDa). Unlike the base TFF Hp purification process, the permeate of the 100 kDa HF filter (Stage 3) was retained on a 50 kDa HF module (P/N: S02-E100-05-N). The new bracket (Stage 4) was subject to constant volume diafiltration with 5x volume PBS and finally concentrated to ~350 mL. A diagram of the entire purification process is shown in FIG. 1.

HPLC-Size Exclusion Chromatography (SEC): Samples from the purification process were separated via HPLC-SEC using a commercial column (4.6 × 300 mm Acclaim SEC-1000, Thermo Fisher Scientific, Waltham, MA) attached to an HPLC system (Dionex UltiMate 3000, Thermo Fisher Scientific, Waltham, MA) as described previously in the literature. (Pires, I. S. & Palmer, A. F. Biotechnol. Prog. 2020, 36, e3010)

Hb Concentration, The concentration of Hb in the samples was measured spectrophotometrically via the Wliiterbotime equations (Winterbourn, C. C. Methods Enzymol. 1990, 186, 265-272).

Gel Electrophoresis: The purity and composition of the protein cocktail was analyzed via SDS-PAGE using commercial equipment (Invitrogen Mini Gel Tank, Thermo Fisher Scientific, Waltham, MA). Samples was prepared according to the manufacturer’s guidelines and as previously described (Pires, I. S. & Palmer, A. F. Biotechnol. Prog. 2020, 36, e3010; and Pires, I. S., et al. Biotechnol. Bioeng. 2020, 117, 125–145).

Total Protein Assay. Total protein was determined via the Bradford assay.

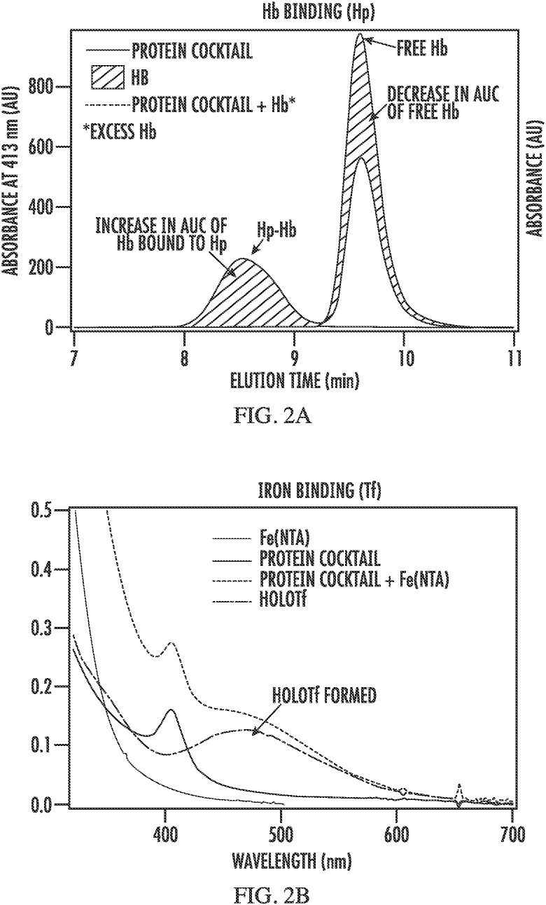

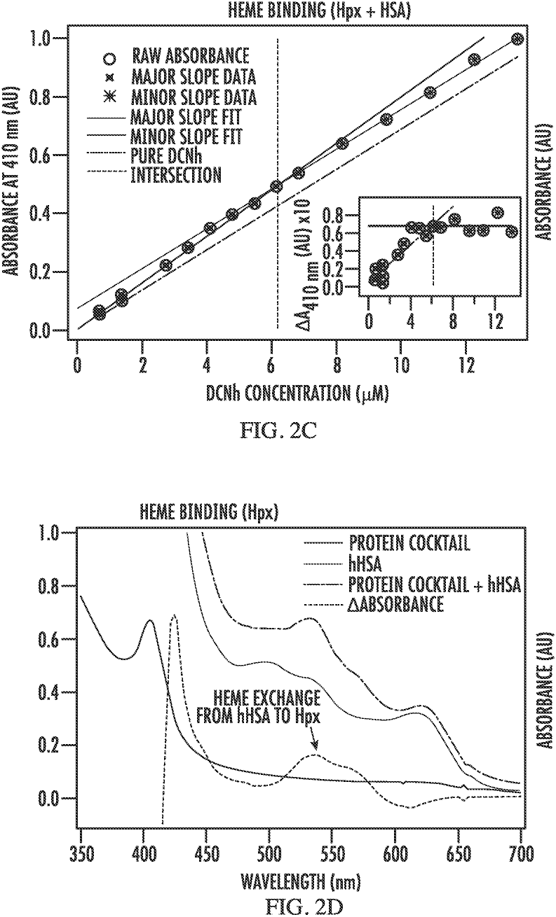

Hb Binding Capacity of Hp: The difference in MW between the Hp-Hb protein complex and pure Hb was used to assess the Hb binding capacity (HbBC) of Hp using HPLC-SEC as previously described (Pires, I. S. & Palmer, A. F. Biotechnol. Prog. 2020, 36, e3010). Briefly, samples containing Hp were mixed with excess Hb then separated via HPLC-SEC. The difference in the area under the curve (AUC) between the pure Hb solution, and the mixture of Hb and Hp was used to assess the HbBC of Hp. A representative HPLC-SEC chromatogram of this assay is shown in FIG. 2A.

Iron Binding Activity: The iron binding capacity (FeBC) of Tf contained in the protein scavenger cocktail was determined via reaction with ferric nitrilotriacetate [Fe(NTA)]. Briefly, the Tf sample was reacted with excess Fe(NTA), and the equilibrium change in absorbance was measured (FIG. 2B). The extinction coefficient of holo- Tf at 465 nm was then used to estimate the concentration of iron bound to Tf (FeBC) (Frieden, E. & Aisen, P. Trends Biochem. Sci. 1980, 5, 10). The holo-Tf concentration was determined based on the 465 nm absorbance of the sample prior to addition of Fe(NTA) (contribution of residual metHb in the sample at 465 nm was estimated based on the sample absorbance at 404 nm, see Meng, F. & Alayash, A. I. Anal. Biochem. 2017, 521, 11–19).

Heme Binding Activity: The heme binding capacity (HemeBC) in the purified protein scavenger cocktail was determined via the dicyanohemin (DCNh) incorporation assay (Pires, I. S., Belcher, D. A. & Palmer, A. F. Biochemistry 2017, 56, 5245-5259). Briefly, the sample was mixed with increasing concentrations of DCNh, and the equilibrium absorbance of the Soret peak maxima was measured. The inflection point in the graph of the equilibrium absorbance versus DCNh concentration was used to determine the saturation point of the heme-binding pockets (FIG. 2C). To determine the heme-binding activity of Hpx individually, the protein cocktail was mixed with excess heme-bound HSA (hi-ISA) and the change in absorbance was used to determine the concentration of heme-Hpx (FIG. 2D) (see WO2019030262).

Trypsin Digest Mass Spectrometry. Protein identification in the protein cocktail was confirmed using trypsin digest nano-liquid chromatography-nanospray tandem mass spectrometry (LC/MS/MS) on a commercial mass spectrometer (Fusion Orbitrap equipped with EASY-Spray™ Sources, Thermo Scientific, San Jose, CA) operated in positive ion mode as described previously in the literature (Pires, I, S. & Palmer, A. F. Biotechnol. Prog. 2020, 36, e3010).

MALDI-TOF-MS. Samples were diluted to 0.5 mg/mL on a protein basis in deionized water. Reduced samples were prepared by adding 0.1 M dithiothreitol to the protein samples. A saturated solution of α-cyano-4-hydroxycinnamic acid matrix was prepared in 50% v/v acetonitrile with 0.1% trifluoroacetic acid. 1 µL of the mixture of the matrix and protein solution was deposited on a matrix assisted laser desorption/ionization (MALDI) plate and analyzed on a MALDI-TOF (time of flight) MS (mass spectrometry) system (Microflex, Bruker, Billerica, MA).

ELISA. The concentration of selected protein components in FIV and in the protein cocktail were quantified via ELISA kits specific for Hp, Tf, HSA, and Hpx according to the manufacturer’s instructions (R&D Systems Catalog #DHAPG0 for Hp, and Eagle BioSciences HTF31-K01 for Tf, HUA39-K01 for HSA, and HPX39-K01 for Hpx).

Viscosity and Colloidal Osmotic Pressure (COP) Measurements. The viscosity of 5% (w/v) HSA and the protein cocktail solution was measured using a cone/plate viscometer (DV-II plus with a cone spindle CPE-40, Brookfield Engineering Laboratories, Middleboro, MA) at a shear rate of 316 s⁻¹, whereas the COP was measured using a colloid osmometer (Wescor 4420, , Logan, UT).

RBC Aggregation. The extent of RBC aggregation of fresh rat whole blood mixed with the test solutions (protein cocktail, saline, 500 kDa dextran and 5% (w/v) HSA) was evaluated in this study. Blood samples were collected into heparinized vacutainers (BD, San Diego, CA) and mixed with 20% by volume of the test solutions. The degree of RBC aggregation was assessed using a photometric rheoscope (Myrenne Aggregometer, Myrenne, Roetgen, Germany) as previously described (Elmer, J., et al. Biotechnol. Prog. 2011, 27, 290-296; and Lee, B. K., et al. Biorheology 2007, 44, 29-35).

Coagulation Studies. Coagulation studies were performed on platelets isolated from citrated (3.2% buffered trisodium citrate, Sigma-Aldrich) rat whole blood and mixed with the protein cocktail solution (20% by volume). Platelets were isolated and aggregation was assessed as previously described subject to stimulation with two agonists: adenosine diphosphate (ADP) and collagen (Oronsky, B., Oronsky, N. & Cabrales, P. J. Cell Mol. Med. 2018, 22, 5076-5082). The effect of the protein cocktail solution was compared to HSA (5% w/v) and Hextend (6% Hetastarch in Lactated Electrolyte solution, Hospira) as control solutions in various platelet functional assays.

Animal Preparation. Animal handling and care followed the NIH Guide for the Care and Use of Laboratory Animals and the experimental protocol was approved by the local animal care committee. Studies were performed in 55 - 65 g male Golden Syrian Hamsters (Charles River Laboratories, Boston, MA) fitted with arterial and venous catheters. For catheter implantation, animals were re-anesthetized with ketamine/xylazine, and a PE-50 catheter with a PE-10 tip was implanted in the right carotid artery and in the jugular vein, secured with 5-0 silk sutures, exteriorized dorsally, and secured to the back of the animals. Hamsters were housed individually post-surgery and allowed to recover one or two days before subjected to experiment.

In vivo Hamster Exchange Transfusion Model. Hamster whole blood was adjusted to 40% hematocrit with 5% HSA. then mixed 4:1 (volume ratio) with a 85 mg/mL solution of the protein cocktail or 6% dextran 70 kDa. 5 mL of the resulting mixture was then mechanically hemolyzed in a sterile closed loop heparin-coated silicone tube circuit with 0.8 mm inner tube diameter using a peristaltic pump running at 100 rpm (5 mL/min) for 12 hours at room temperature. After mechanically hemolyzing the blood mixture, the plasma from the hemolyzed mixture was separated via centrifugation and the supernatant stored at -80° C. For in vivo testing, hemolyzed blood plasma filtered through a 0.2 µm filter was used to perform an isovolumic exchange transfusion of 5% of the animal’s total Hb mass at a rate of 100 µl/min. Animals were euthanized two hours post transfusion to collect the blood, liver, heart, spleen, and kidneys for analysis. Organ tissue was processed for inflammatory and organ damage markers as previously described (Muller, C. R., et al. Transfusion 2021, 61, 212-224).

Statistical Analysis. All data are represented as mean values ± SD. Data were compared using a one-way ANOVA with Tukey’s multiple comparisons test for parametric data using commercial statistical software (GraphPad Prism 8.1, GraphPad Software Inc., San Diego, CA). Changes were considered significant if p values were less than 0.05.

### RESULTS

### Protein Cocktail Purification and Composition

The results from three batches following the protein cocktail purification process outlined in FIG. 1 are summarized in Table 1.

Table 1: Summary of the scavenging protein cocktail composition, concentration, and yield. Percentage composition was assessed via the activity binding assays. HbBC = Hb-binding capacity. HemeBC = heme-binding capacity. FeBC = iron-binding capacity. All molar concentration values are provided on a globin/iron basis (i.e. tetrameric Hb contains four globin/iron equivalents, while both heme and iron contain one iron equivalent). Error is based on the standard deviation of three independent batches.

| | Total (g) | ± | Concentration (mg/mL) | ± | Yield (%) | ± |
|---|---|---|---|---|---|---|
| Protein | 61.9 (100%) | 9.4 | 171 | 21 | 50 | 4 |
| Hp * | 5.97 (10%) | 0.12 | 16.5 (16^{§}) | 0.7 | 49 | 3 |
| Hpx | 2.21 (4%) | 0.42 | 6.1 (8.8^{§}) | 0.9 | (52^{§}) | (2) |
| HSA^{†} | 28.7(42%) | 4.3 | 79.1 (75^{§}) | 10.1 | (88^{§}) | (10) |
| apoTf | 18.2 (29%) | 2.7 | 50.1 | 6.03 | - | - |
| Tf^{‡} | 3.05(34%) | 0.17 | 58.6 (63^{§}) | 5.61 | (81^{§}) | (9) |
| Other | 6.12 (10%) | 2.4 | 16.9 | 6.1 | - | - |
| | | Concentration (mM) | | ± | | |
| | | HbBC | 0.61 | 0.03 | | |
| | | HemeBC | 1.22 | 0.16 | | |
| | | FeBC | 1.25 | 0.15 | | |

* Hp determined assuming a 1:1.65 mass binding ratio of Hb:Hp2-2
^{†} HSA determined by the total heme binding capacity excluding the contribution from Hpx
^{‡} Total Tf determined via the non-Hb contribution from the absorbance at 460 nm
^{§} Concentration and yield determined from ELISA kits

As shown in Table 1, each 500 g batch of FIV yielded more than 60 g of a concentrated protein cocktail composed primarily of HSA and Tf with ~10% and 5% of Hp and Hpx, respectively. ELISA results concurred with the activity binding assays, demonstrating that the proposed series of activity assays (FIGS. 2A-2D) was capable of accurately quantifying the different protein species in the sample.

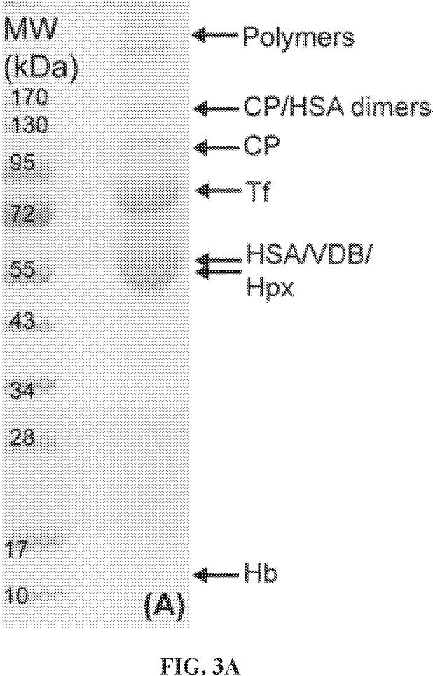

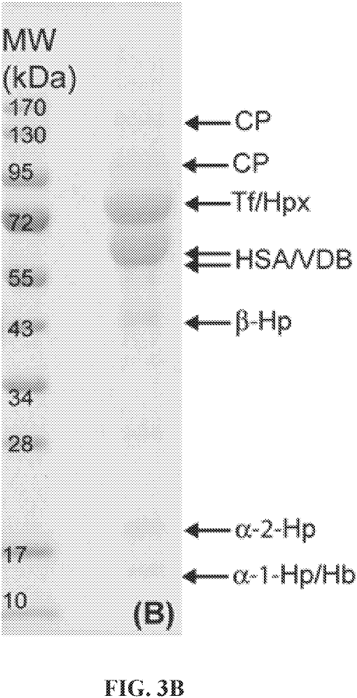

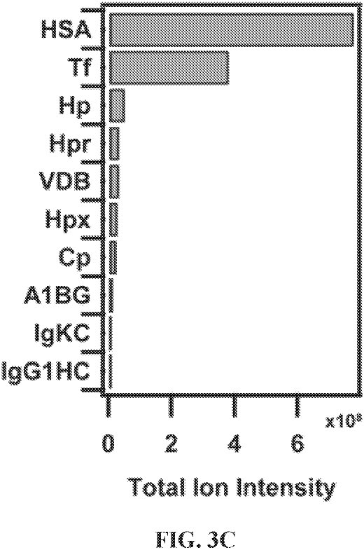

To further analyze the composition of the protein cocktail, samples were analyzed via SDS-PAGE, trypsin digest mass spectrometry (MS), and MALDI-TOF MS. The results of representative samples are shown in FIGS. 3A-3C.

As expected, HSA, Tf, Hpx, Hp were present in the SDS-PAGE (FIGS. 3A and 3B) and identified in the trypsin digest MS analysis (FIG. 3C). The detection of haptoglobin-related protein (Hpr) was likely due to the high sequence identity of Hpr compared to the Hp1-1 phenotype (Pires, I. S. & Palmer, A. F. Biotechnol. Prog. 2020, 36, e3010). Furthermore, ceruloplasmin (Cp) and vitamin-D binding protein (VDB) were also detected in the trypsin digest MS with similar ion intensities as Hpx. Thus, it would be expected that these components had similar mass composition to Hpx (~5%).

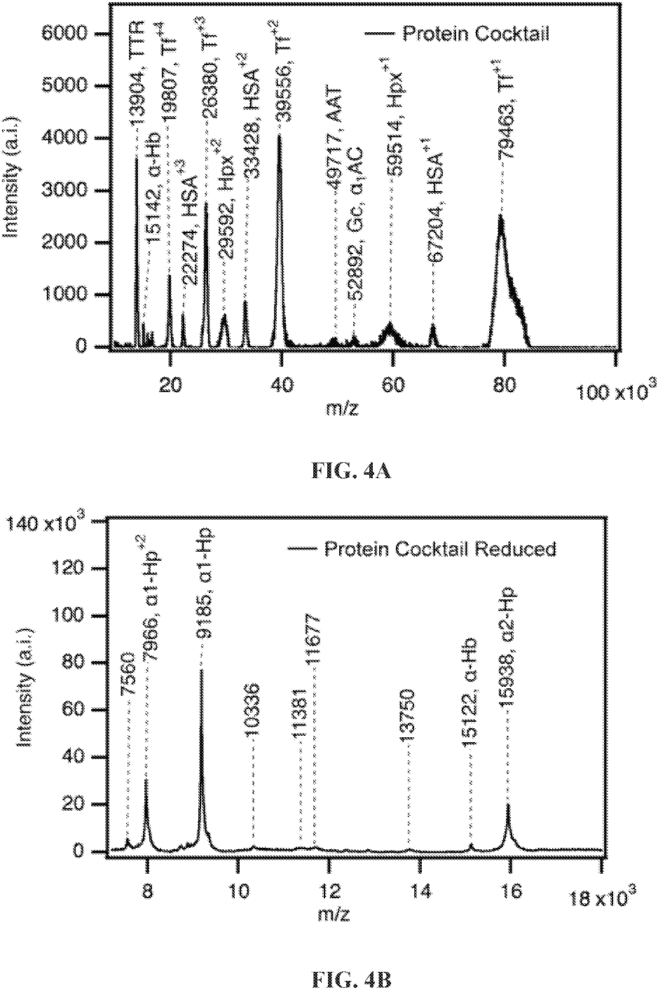

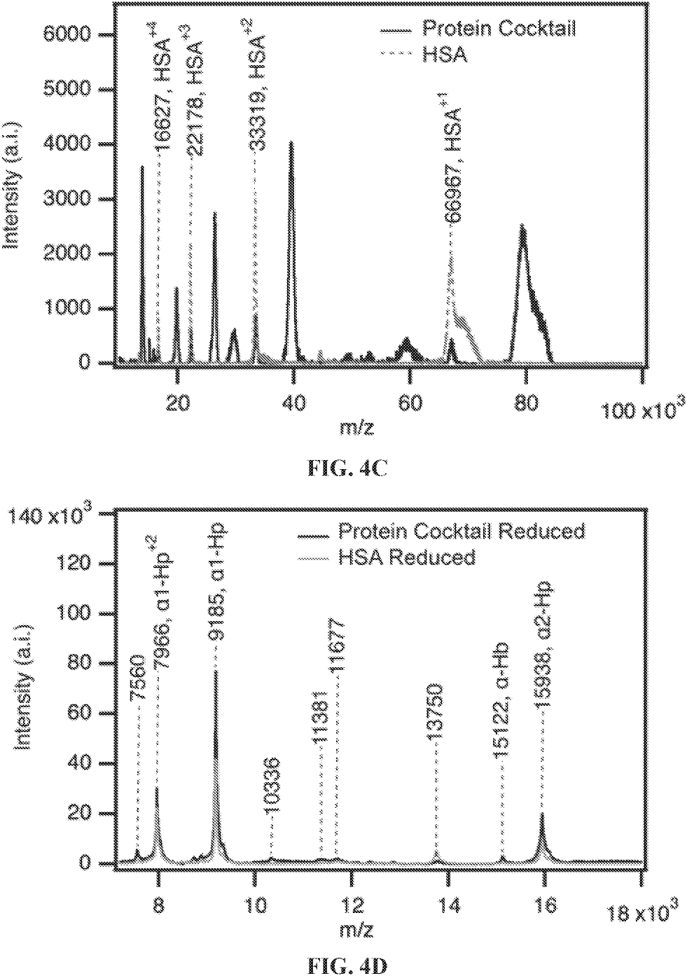

Moreover, the various peaks in the MALDI-TOF MS spectra (FIGS. 4A-4D) of the protein cocktail confirmed the presence of the expected protein species. Notably, MALDITOF MS data cannot be used as a quantitative method for assessment of composition as different proteins have different ionization efficiencies (Hortin, G. L. & Remaley, A. T. Clin. Proteomics 2006, 2, 103-116). However, densitometric analysis of SDS-PAGE can provide quantitative insights into protein composition, and the results are shown in Table 2.

TABLE 2

Composition of the scavenging protein cocktail based on SDS-PAGE densitometric analysis. Error is based on the standard deviation of three independent batches.

| NON-REDUCED | | | REDUCED | | |
|---|---|---|---|---|---|
| Species | Composition | +/- | Species | Composition | +/- |
| HSA/Hpx/VDB | 47.7% | 1.7% | Tf/Hpx | 42.4% | 3.1% |
| Tf | 31.2% | 1.2% | HSA/VDB | 40.3% | 3.4%. |
| Polymers (Hp) | 15.2% | 3.0% | β-Hp | 4.8% | 0.1% |
| Cp (135 kDa) | 2.4%, | 0.0% | α-2Hp | 3.3% | 0.5% |
| HSA dimer/ Cp (115 kDa) | 2.4% | 0.1% | α-1Hp/Hb | 3.0% | 1.0% |
| Other | 1.1% | 0.0% | Cp (115 kDa) | 1.9%, | 0.0% |
| | | | Other (>140 kDa) | 1.7% | 0.7% |
| | | | Cp (135 kDa) | 1.7% | 0.0% |
| | | | Other (~28 kDa) | 1.6% | 0.4% |
| | | | β-2 glycoprotein | 0.7% | 0.1% |

Abbreviations: HSA: human serum albumin; Hpx: hemopexin; VDB: vitamin-D binding protein (Gc~globulin); Tf: transferrin; Hp: haptoglobin, Cp: ceruloplasmin; Hb: hemoglobin

Densitometric analysis results agreed with the activity binding assay results where >90% of the protein cocktail was composed of four major proteins (HSA, Hpx, Hp, and Tf). Cp was also noticeable on the SDS-PAGE with ~4% mass composition. Hpx could only be partially estimated via SDS-PAGE analysis as it alters its apparent MW when reduced. Comparing the percent composition of Tf and the HSA band before and after reduction showed a 7-9% change, indicating a similar composition as the estimation determined via the heme-binding assay (~5%).

### Biophysical and Biochemical Properties of the Protein Cocktail

In vivo use of the protein cocktail requires understanding of the physicochemical properties of the mixture and its effects when administered into blood. Thus, the colloidal osmotic pressure (COP) and viscosity of the protein cocktail were assessed and compared to that of commercial 5% (w/v) HSA. The results are shown in .

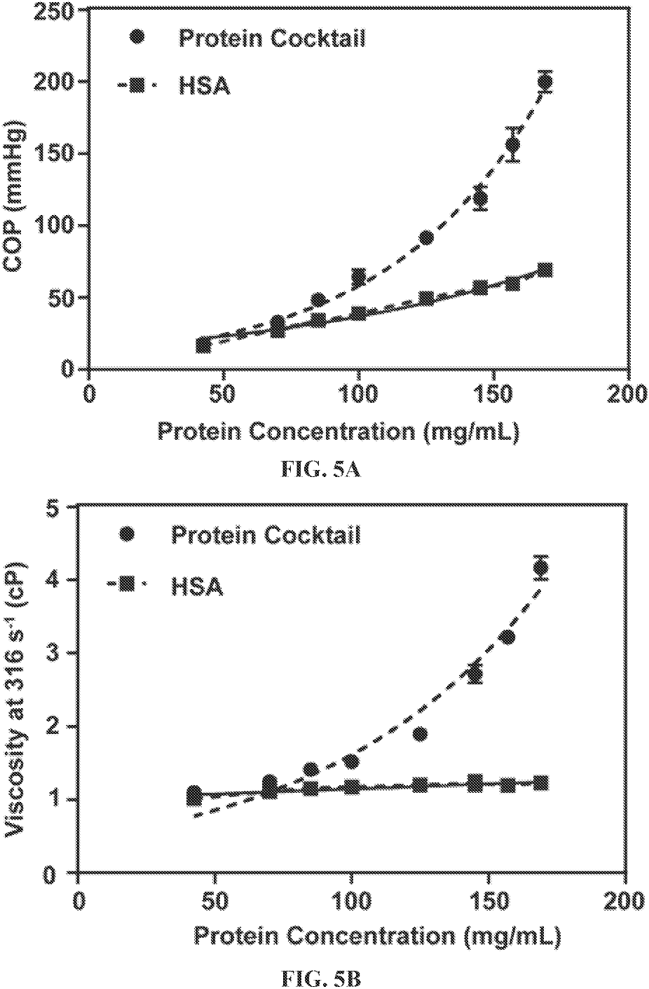

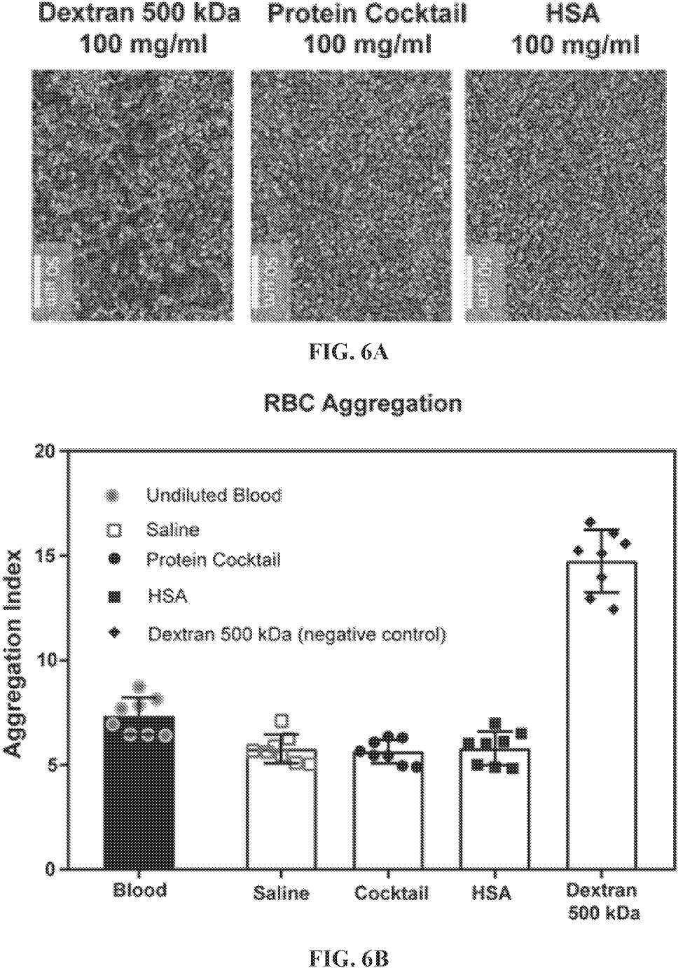

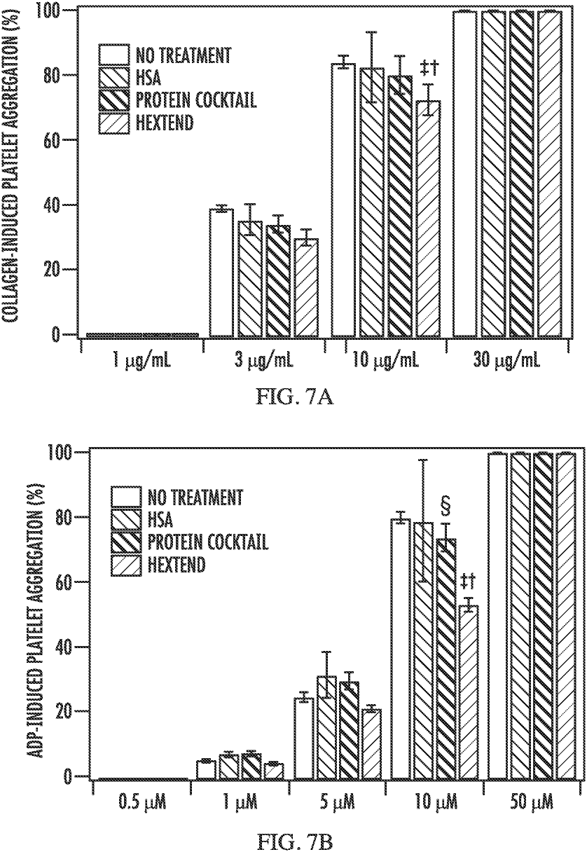

As shown in FIGS. 5A-5B, the protein cocktail had similar COP and viscosity to HSA at concentrations lower than 75 mg/mL. However, at high protein concentrations, both the COP and viscosity of the cocktail showed a highly non-linear increase with protein concentration reaching values higher than pure HSA. Next, the protein cocktail was mixed with whole blood and platelet rich plasma to assess its effects on RBC and platelet aggregation. The results are shown in FIGS. 6A-6B and 7A-7B.

Based on the results shown in FIGS. 6A and 6B, unlike 500 kDa dextran, the protein cocktail did not lead to RBC aggregation as its aggregation index was similar to that of blood mixed with saline or HSA. Furthermore, as shown in FIGS. 7A and 7B, the collagen platelet aggregation test showed that the protein cocktail did not lead to significant platelet aggregation inhibition compared to the control or HSA. On the other hand, Hextend was the only material tested that significantly impaired platelet aggregation.

### In Vivo Efficacy of the Protein Cocktail at Hemolysis Treatment

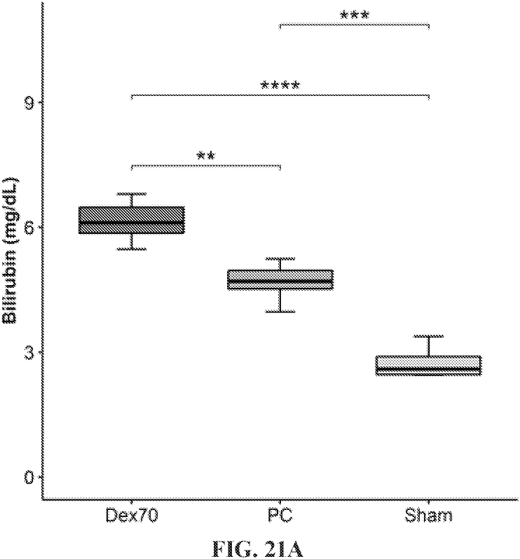

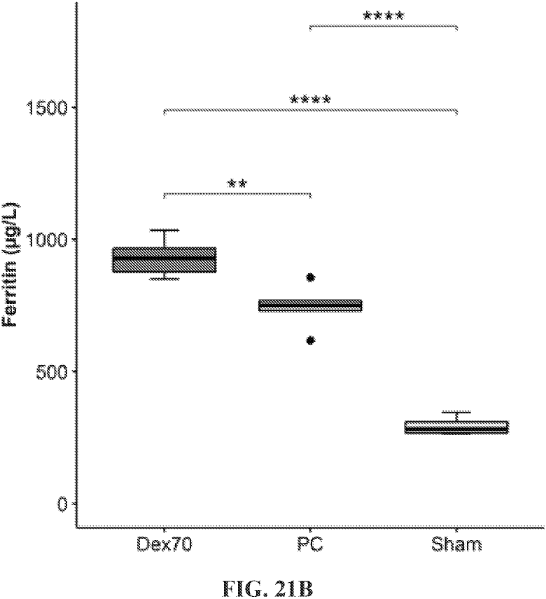

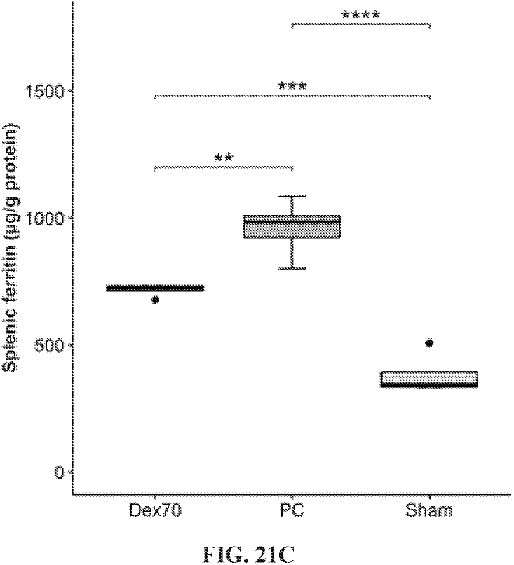

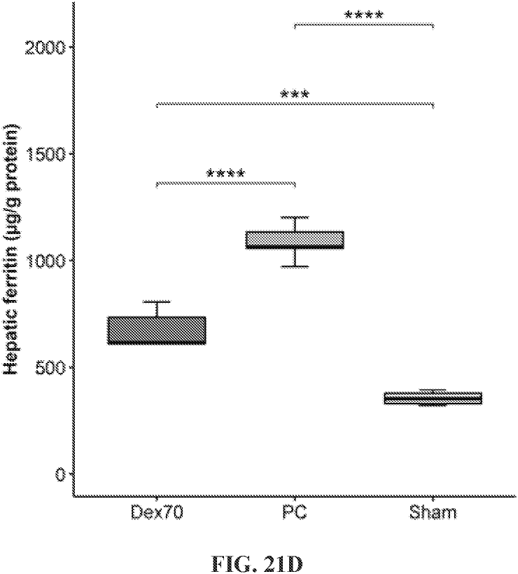

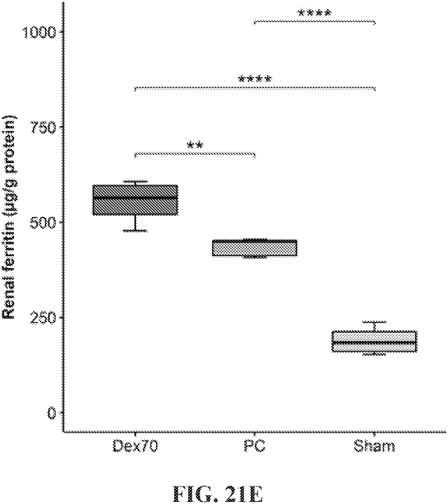

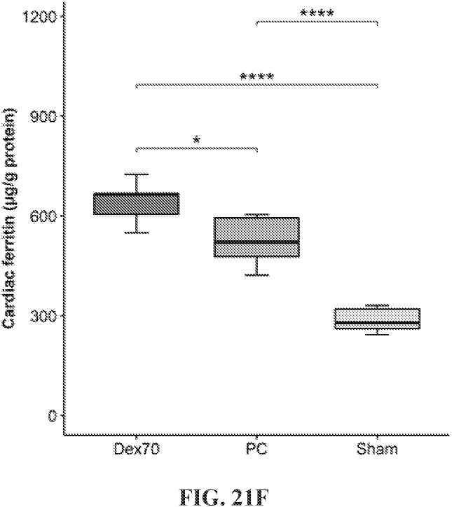

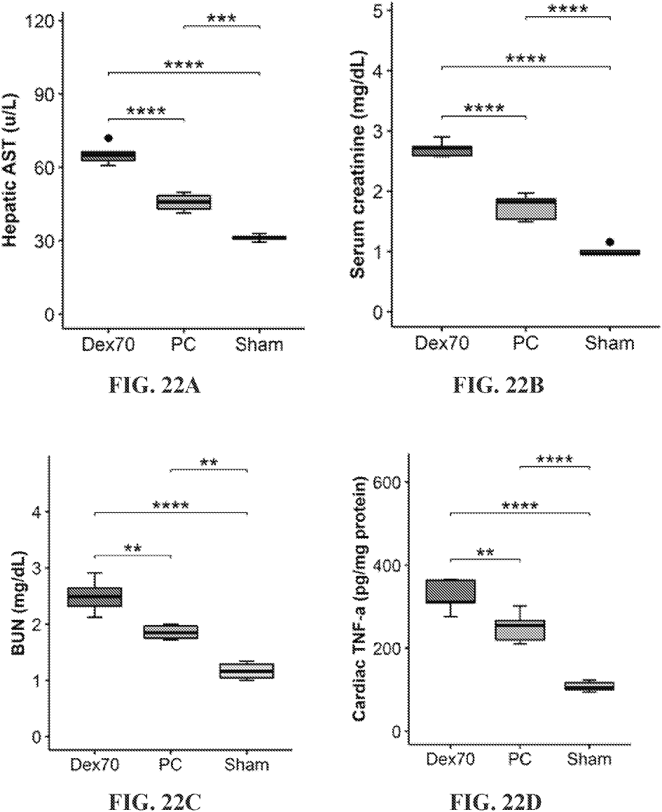

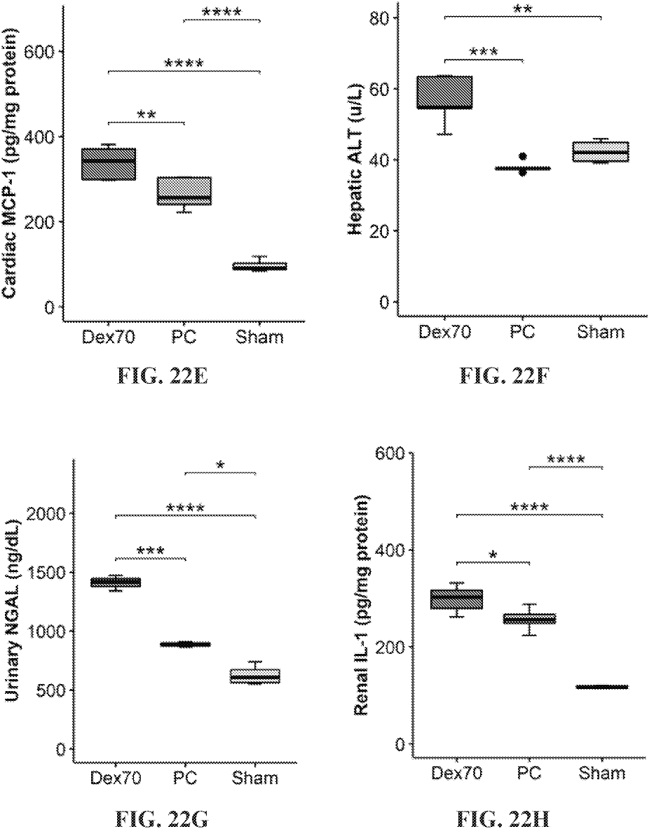

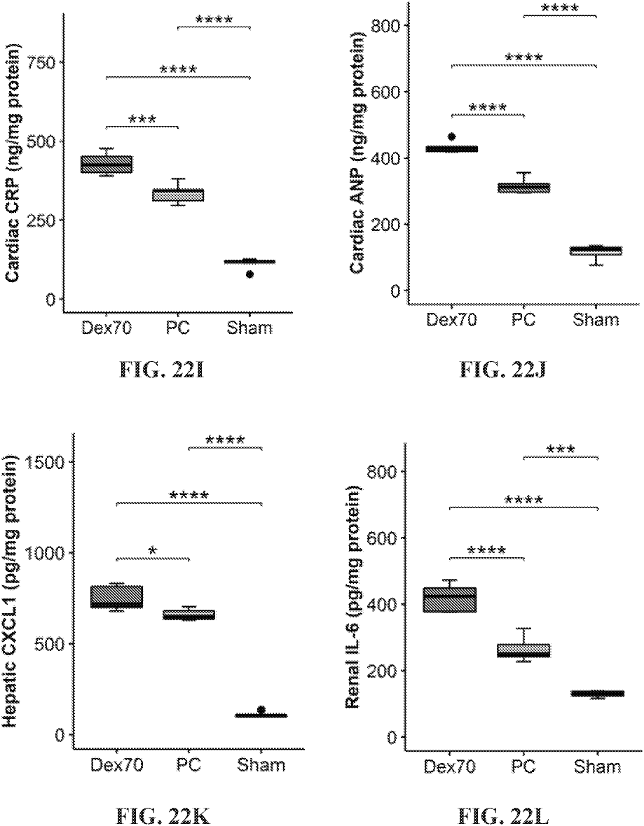

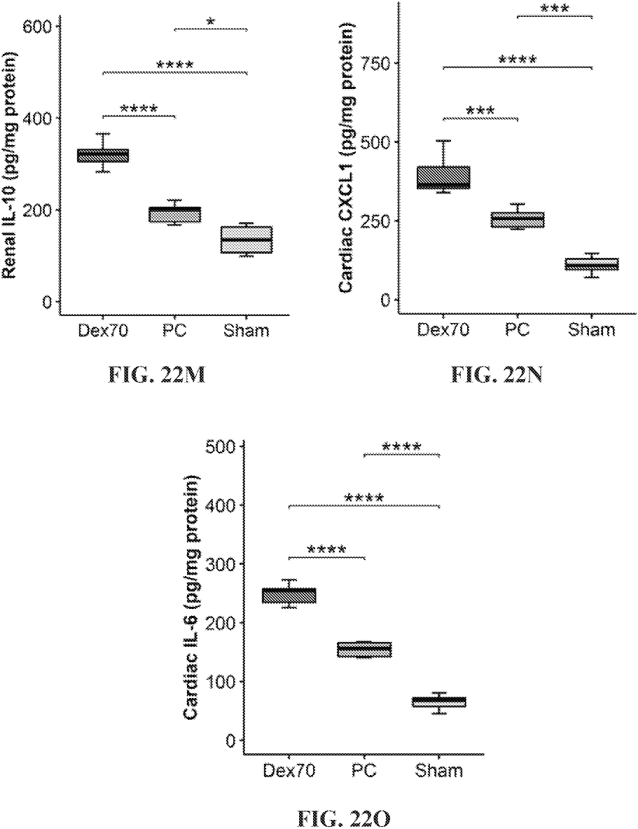

The promising properties of the isolated protein cocktail in hemolysis treatment was tested in vivo by administering mechanically hemolyzed blood plasma mixed with either the protein cocktail or dextran 70 kDa (Dex70) via exchange transfusion at 5% of the animal’s total Hb mass. Dex70 was chosen for comparison given its improved plasma expansion properties compared to HSA, potential anti-inflammatory effects, and interest as a pump-priming fluid (Gombocz, K., et al. Crit, Care 2007 11, R87; and Barbu, M., et al. Ann. Thorac. Surg. 2020, 110, 1541-1547). Ferritin levels in the heart, kidney, liver and blood were measured and markers of tissue inflammation or injury were also assessed. As shown in FIGS. 21A-21F, two hours post-exchange transfusion, there was a significantly altered iron distribution in the animals. Administration of the protein cocktail with hemolyzed blood plasma led to lower circulating levels of bilirubin and ferritin (FIGS. 21A-21B) in transfused animals. This iron was directed to the proper clearance organs responsible for iron metabolism such as the spleen and liver (FIGS. 21C-21D) and prevented from accumulating in iron-sensitive organs such as the kidneys and heart (FIGS. 21E-21F). To further assess organ toxicity from transfusion of the hemolyzed blood plasma, inflammatory and injury markers for renal, hepatic and cardiac tissues were measured and the results are shown in FIGS. 22A-22O.

As shown in FIGS. 22A-22O, inflammatory markers in renal, hepatic and cardiac tissues were significantly reduced approaching baseline levels in healthy animals. Notably, although liver ferritin was elevated, the assayed markers of liver injury were reduced compared to Dex70 indicating detoxification of the iron-containing molecules released from hemolysis.

### Discussion

Comprehensive treatment of hemolysis requires the scavenging of three toxic species: cell-free Hb, heme and iron. Based on the results presented here, the isolated scavenging protein cocktail was capable of binding to these three molecules via Hp (~10%), HSA. (~40%), Hpx (~5%) and Tf (~35%). Furthermore, the protein cocktail was also composed of Cp (~5%) and VDB (~5%), which can help mitigate hemolysis-associated toxicity. Moreover, the favorable hemocompatibility, colloidal osmotic pressure (COP) and viscosity properties indicate potentially favorable outcomes in vivo. Indeed, preliminary in vivo studies demonstrated that that this protein cocktail could reduce hemolysis-associated iron toxicity.

The protein cocktail contained a large fraction of HSA. Although not as efficient as either Hpx or Tf, HSA binds to both free heme and free iron, reducing their oxidative toxicity and serving as a reservoir until Hpx and Tf can deliver these molecules to their respective clearance receptors (Loban, A., Kime, R. & Powers, H. Clin. Sci. (Lond). 1997, 93, 445-51; Taverna, M., Marie, A.-L., Mira, J.-P. & Guidet, B. Ann. Intensive Care 2013, 2013, 3, 4; Tolosano, E. & Altruda, F. DNA Cell Biol. 2002, 21, 297-306; and Smith, A. & McCulloh, R. J. Front. Physiol. 2015, 6, 187). Furthermore, the large HSA fraction in the protein cocktail indicate its potential to serve as a plasma expander with hemolysis mitigating properties. FIG. 8 summarizes the role of the major components of the protein cocktail in reducing hemolysis damage.

The other identified components of the protein cocktail - Cp and VDB - play secondary roles in reducing hemolysis-induced toxicity. Cp is a ferroxidase that catalyzes the oxidation of Fe²⁺ into Fe³⁺ and for stabilization of ferroportin (cellular iron exporter) (Ramos, D., et al. PLoS One 2016, 11, e0149516; and De Domenico, I., et al. EMBO J. 2007, 26, 2823-31). Notably, the oxidation of iron to Fe³⁺ is required for iron binding to Tf (iron transport) or ferritin (iron storage) (Ramos, D., et al. PLoS One 2016, 11, e0149516; de Silva, D. & Aust, S. D. Arch. Biochem. Biophys. 1992, 298, 259-264; Samokyszyn, V. M., Miller, D. M., Reif, D. W. & Aust, S. D. J. Biol. Chem. 1989, 264, 21-26; Silva, D. M. de & Aust, S. D. Can. J. Physiol. Pharmacol. 1993, 71, 715-720; and Hellman, N. E. & Gitlin, J. D. Annu. Rev. Nutr. 2002, 22, 439-458). On the other hand, VDB, also known as Gc-globulin, scavenges actin which is another toxic species released during hemolysis or tissue damage (Smith, D., et al. Blood 1988, 72, 214-218; Peddada, N., Sagar, A. & Garg, R. Med. Hypotheses 2012, 78, 203-210; Piktel, E., et al. Int. J. Mol. Sci. 2018, 19, 2516, Meier, U., et al. Clin. Chem. 2006, 52, 1247-1253; and Chun, R. F. Cell Biochem. Funct. 2012, 30, 445-456).

In addition to reducing hemolysis toxicity, the scavenging protein cocktail has promising viscous and COP properties. This characteristic may be due to the large MW components of the cocktail, which could promote protein crowding via depletion forces (Mitchison, T. J. Mol. Biol. Cell 2019, 30, 173-180). Blood viscosity is an important factor that regulates the responses of the cardiovascular system, as it affects shear stress and activates the synthesis of vascular relaxation mediators such as nitric oxide (NO) (Tsai, A. G., et al. Am. J. Physiol. Circ. Physiol. 2005, 288, H1730-H1739). NO is a critical regulator of basal blood vessel tone and vascular homeostasis, antiplatelet activity, modulation of endothelial and smooth muscle proliferation, and adhesion molecule expression. From a rheological standpoint, an acute decrease in hematocrit paired with a decrease in plasma viscosity is highly detrimental. However, by using the protein cocktail presented here, the high viscosity of the cocktail could partially preserve vascular endothelial shear stress. Studies in the microcirculation using hemodilution have shown that high viscosity solutions significantly improved microvascular function and organ blood flow compared with low viscosity solutions (Tsai, A. G., et al. Am. J. Physiol. Circ. Physiol. 2005, 288, H1730–H1739). Moreover, there are physiological implications of the nonlinear COP of the protein cocktail as a function of protein concentration, which indicates the potential of the protein cocktail to expand the blood volume beyond the volume infused, by pulling extravascular fluid into the intravascular space from the tissue space. Similarly, reducing RBC aggregation can thin the RBC-poor plasma layer near the vascular wall, which positively increases vascular endothelial shear stress. Lastly, the positive hemocompatibility (lack of RBC aggregation) and the preservation of platelet function are very positive properties of the protein cocktail solution and support its potential application in transfusion medicine.

Although plasma also contains the same proteins in the protein cocktail, there are safety considerations that favor use of the processed protein cocktail. Without the presence of immunoglobulins, the protein cocktail serves as a universally transfusable solution. This expands the source of plasma that can be used to purify the protein cocktail, since only 4% of the U.S. population has type AB blood (universal plasma donor) (Nascimento, B., et al. Crit. Care 2010, 14, 202). Furthermore, immunoglobulins are known to increase the risk of transfusion-related acute lung injury, which is considered the leading cause of transfusion-related mortality (Kim, J. & Na, S. Korean J. Anesthesiol. 2015, 68, 101-105; and Miller, T. E. Perioper. Med. 2013, 2, 13). Moreover, due to the ethanol precipitation steps used to produce FIV and the extensive nanofiltration used to isolate the protein cocktail, the risk of transmission of blood-borne infectious agents is greatly minimized compared to plasma administration.

In addition to its improved safety profile, the concentrations of desired hemolysis scavenging proteins in the protein cocktail are enhanced compared to plasma. The protein concentration in human plasma ranges from 60-80 mg/mL with a composition of approximately 50% HSA, 5% Tf, 2% Hp, 1 % Hpx and less than 1% of Cp or VDB (Li, C., et al. Sci. Rep. 2016 6, 24329; and Kramer, G., et al. PLoS One 2015, 10, e0140097). Therefore, the composition of Tf, Hp, Hpx, Cp and VDB in the protein cocktail all had approximately a five-fold or greater increase compared to plasma. Lastly, from a fluid resuscitation perspective, a potential disadvantage of the protein cocktail relative to plasma may be the lack of coagulation proteins. However, the benefit of plasma resuscitation maybe associated with its HSA content and not the presence of coagulation factors (Kheirabadi, B. S., et al. J. Trauma Acute Care Surg. 2016, 81, 42-49).

Future generations of the protein cocktail may have altered composition by supplementing with additional proteins or adding additional processing steps before or after the filtration system presented here such as precipitation with ammonium sulfate or chromatographic techniques (Raoufinia, R., et al. Adv. Pharm. Bull.2016, 6, 495-507). However, these extra processing steps would increase manufacturing costs and complexity.

### Conclusion

Approximately 60 g of a protein cocktail was isolated from 500 g of FIV via TFF, yielding a protein mixture with Hb, heme and iron binding capability. Interestingly, the protein cocktail showed a non-linear concentration dependence with respect to viscosity and COP, which are advantageous properties for a plasma expander. Furthermore, the protein cocktail did not elicit red blood cell aggregation nor inhibit platelet aggregation in vitro which further demonstrates its potential use in transfusion medicine. In vivo studies confirmed the reduction of hemolysis-mediated toxicity by improving iron transport and reducing cardiac, hepatic and renal tissue damage.

Taken together, this example presents a simple and effective method to purify and characterize a blood-compatible protein cocktail capable of scavenging free iron, free heme, and cell-free Hb for possible treatment of states of hemolysis and as a new generation plasma expander for use in transfusion medicine.

### Example 2. Effect of a Protein Scavenging Cocktail on Blood Coagulation and Resuscitation From Hemorrhagic Shock

Hemorrhagic shock represents the leading cause of potentially preventable deaths on the battlefield. Successful management of hemorrhagic shock in the field requires both achieving hemostasis and restoration of blood volume to preserve microcirculatory O₂ transport. Hydroxyethyl starch (HES) solutions were, until recently, the gold standard resuscitation solution when blood was not readily available, such as in the field. However, it was recently discovered that HES solutions result in coagulopathies that impair proper hemostasis, and as such their use declined. The need for alternatives for field plasma expansion is clear. Described herein are albumin-based plasma expanders (also referred to herein as protein scavenging cocktails), that can also include protein(s) that assist in coagulation (such as ceruloplasmin’s role in normalization of endothelial function and platelet activity, and prevention of uncontrolled coagulation via Gc-globulin actin scavenging properties), as well as proteins that scavenge toxic free iron, heme and cell-free hemoglobin (transferrin, hemopexin, and haptoglobin). In this example, the impact of HES and the protein scavenging cocktail on blood coagulation are evaluated, and their efficacy during resuscitation from hemorrhagic shock are examined.

Coagulation parameters were measured using fresh rat whole blood mixed with 20% by volume of the test solutions composed of either HSA (5% w/v), Hextend (6% Hetastarch in Lactated Electrolyte solution, Hospira), or the protein cocktail (85 mg/mL).

To study the efficacy of these solutions during hemorrhagic shock, golden Syrian hamsters instrumented with a dorsal window chamber, and catheters in the left common carotid artery were hemorrhaged for 50% of their blood volume (estimated as 7% of body weight) and resuscitated with 25% of their blood volume with fresh blood, Hextend [colloidal plasma expander composed of hydroxyethyl starch (HES)], or the protein scavenging cocktail. Shock and resuscitation were monitored for 1 hour each. Microvascular perfusion was characterized by vessel diameter, flow, functional capillary density, and pO₂ in arterioles, venules, and extravascular tissue. Systemic variables, such as mean arterial pressure (MAP), heart rate (HR), pO₂, pCO₂, pH, glucose, and lactate were also monitored.

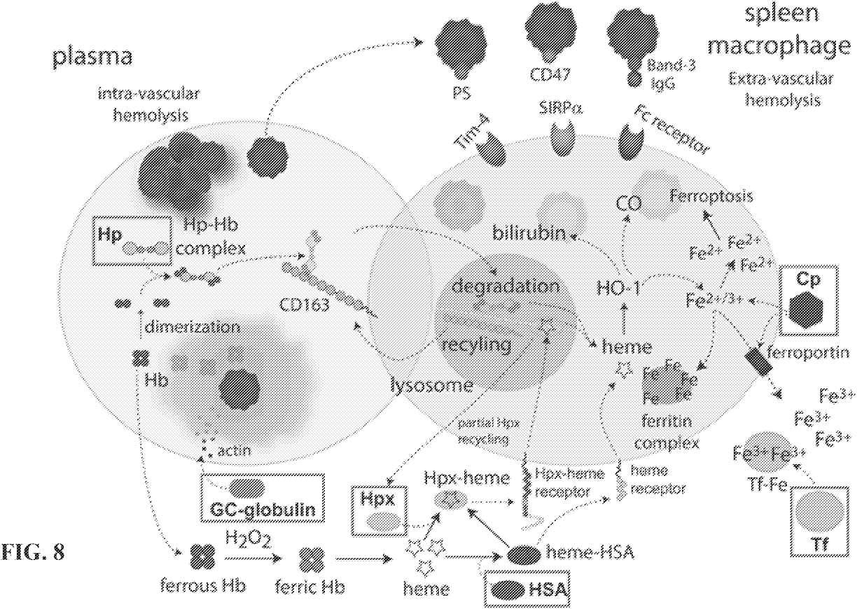

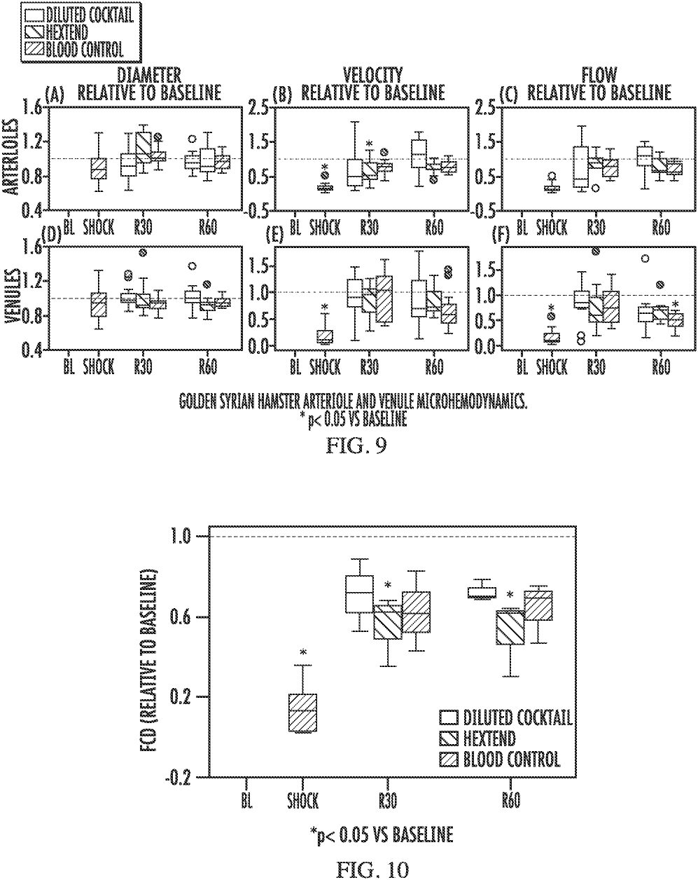

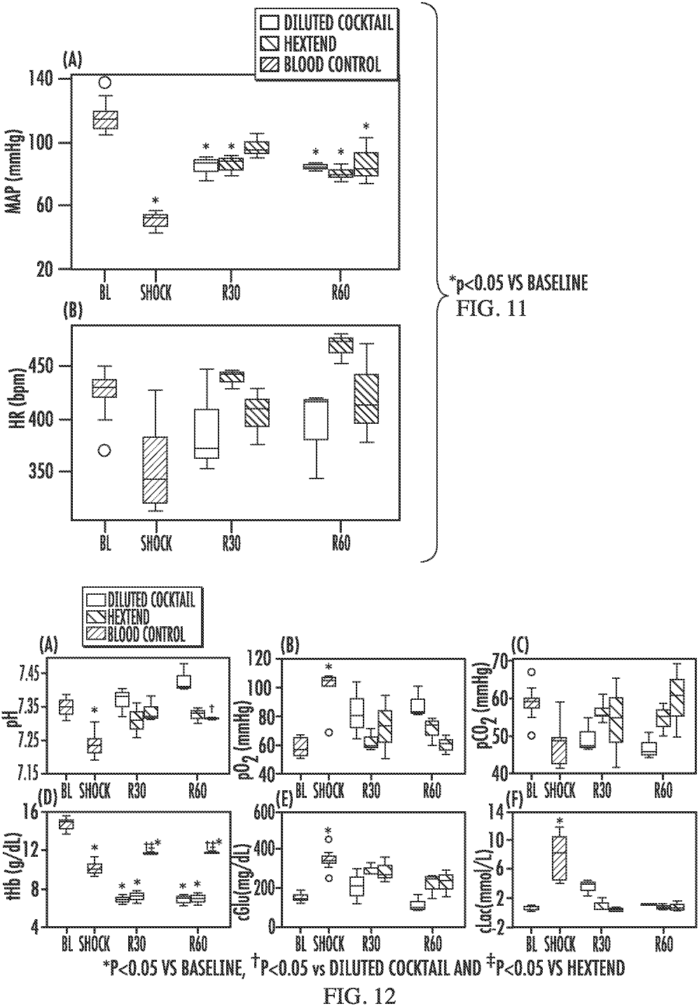

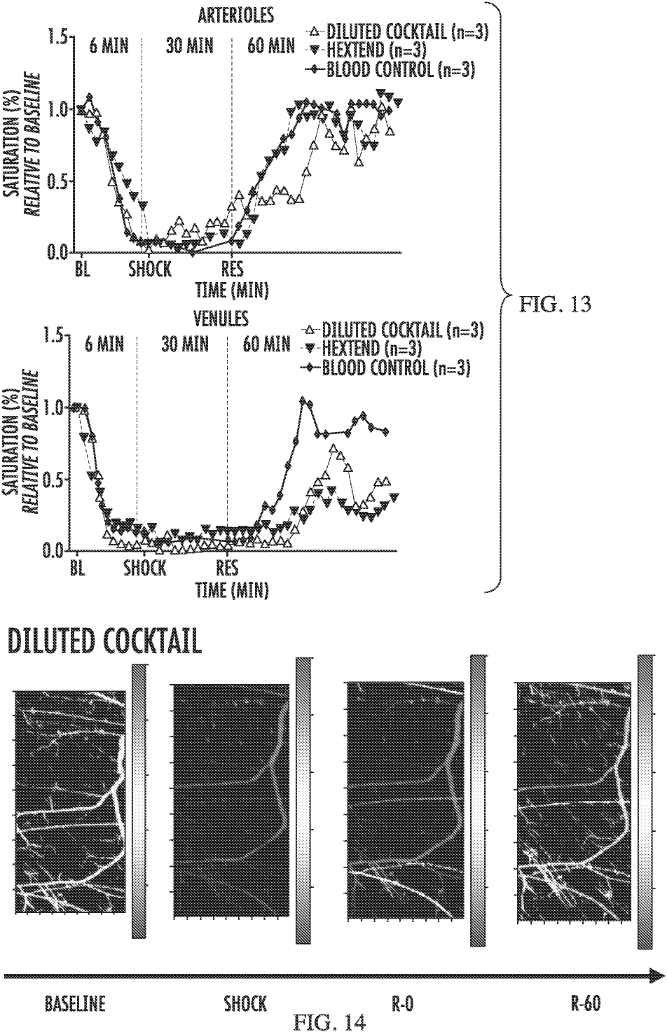

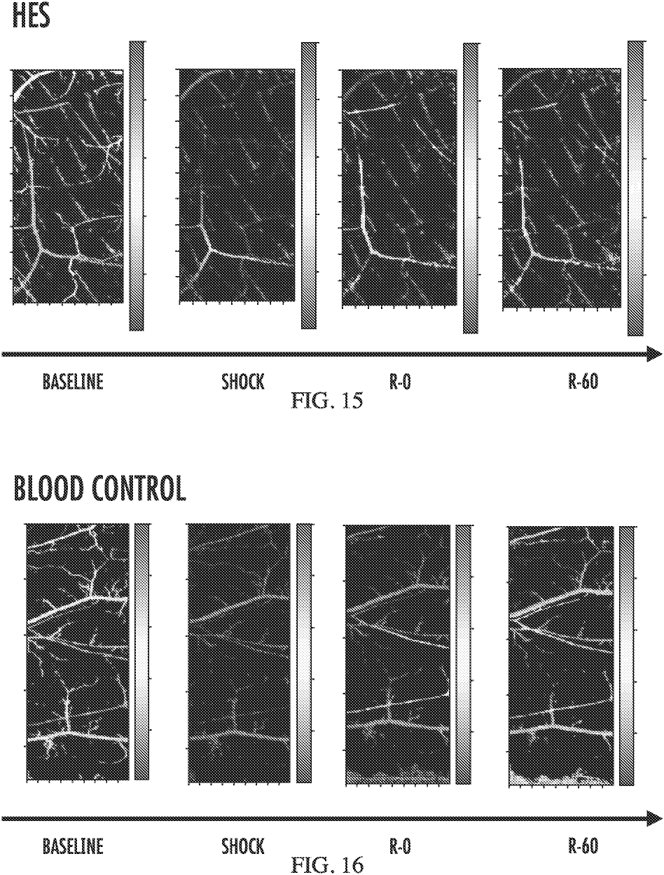

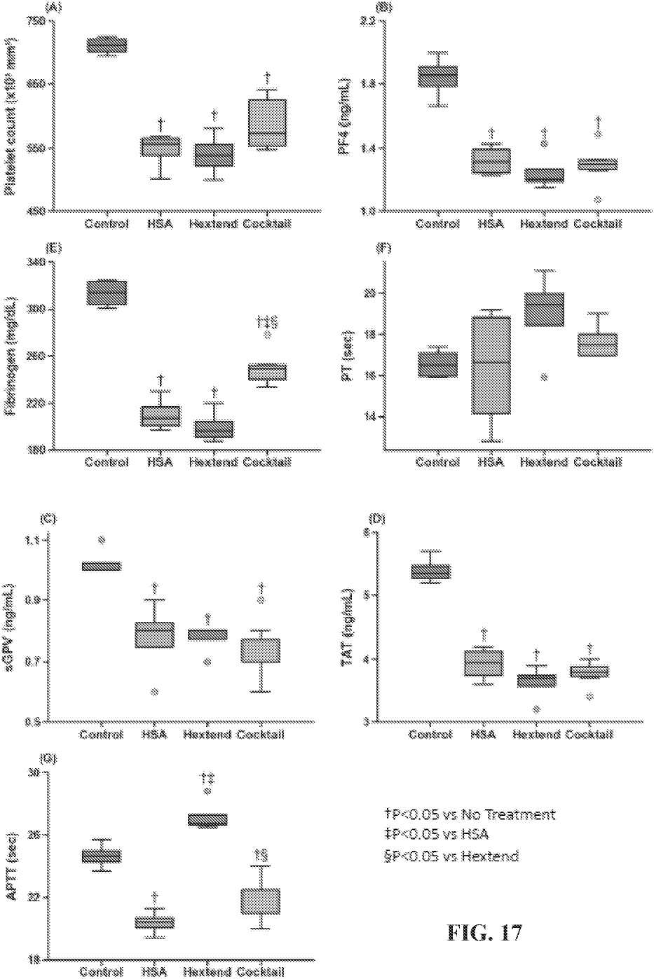

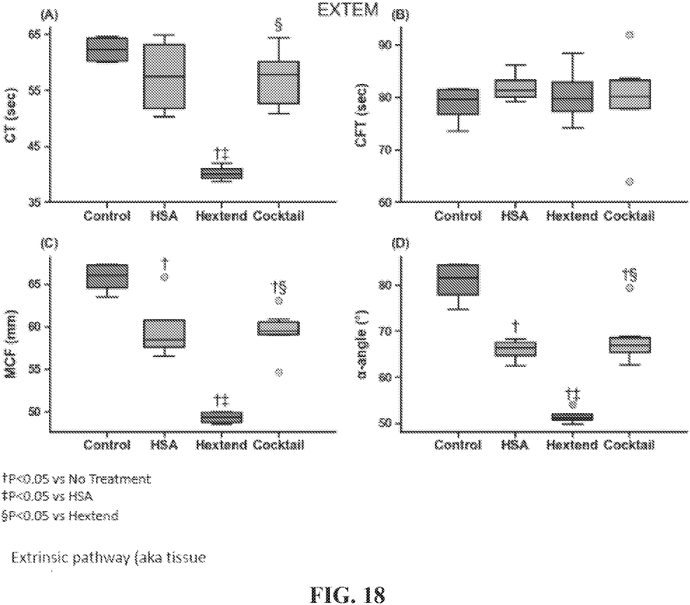

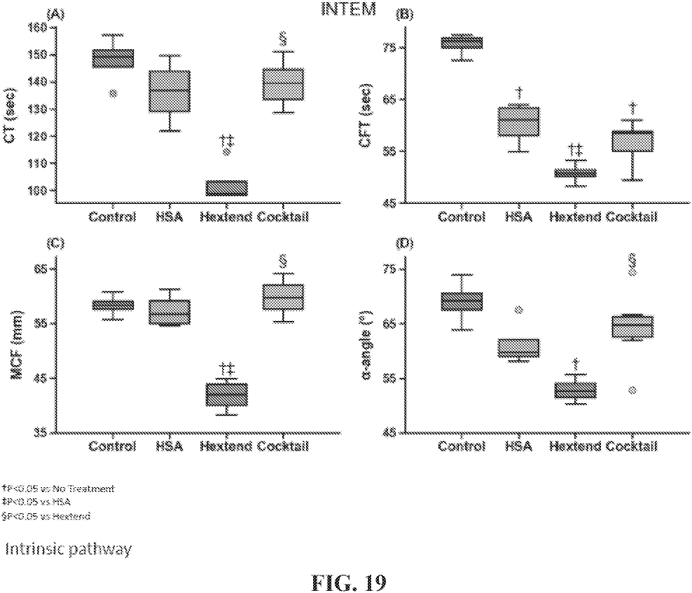

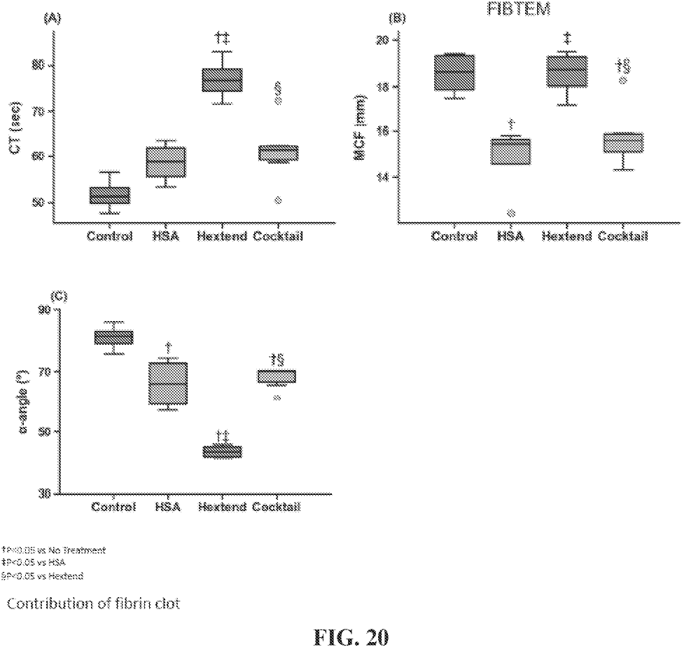

The results of these initial studies are detailed in FIGS. 9-20. Briefly, the protein scavenging cocktail did not result in coagulopathies, but presented slightly lower clot strength and clotting time compared to un-bled controls, which was to be expected with the dilution of platelets and RBCs. However, HES resulted in significantly decreased clotting time and clot strength compared to controls and the protein scavenging cocktail. Additionally, HES transfusion decreased platelet aggregation in response to both collagen and ADP, which could decrease the strength of the platelet plug and increase the time for said plug to form. The use of the protein scavenging cocktail improved microvascular blood flow and oxygen transport during shock with the capability to lessen the burden of potential hemolysis that can occur during various forms of shock. Notably, resuscitation with blood or the protein cocktail lead to a nonsignificant change in functional capillary density (FCD) while HES was significantly lower than baseline (FIG. 10).

A number of embodiments of the disclosure have been described. Nevertheless, it will be understood that various modifications may be made without departing from the spirit and scope of the invention. Accordingly, other embodiments are within the scope of the following claims.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method for treating plasma protein imbalances or depletion in a subject in need thereof comprising administering to the subject a therapeutically effective amount of a protein composition comprising:
from 5% by weight to 99% by weight haptoglobin, based on the total weight of all proteins in the composition; and
from 1% by weight to 95% by weight transferrin, based on the total weight of all proteins in the composition.

2. The method of claim 1, wherein composition comprises from 5% by weight to 60% by weight or from 5% by weight to 25% by weight haptoglobin, based on the total weight of all proteins in the composition.

3. The method of claim 1, wherein the composition comprises from 1% by weight to 50% by weight or from 30% by weight to 40% by weight transferrin, based on the total weight of all proteins in the composition.

4. The method of claim 1, wherein the composition further comprises from 1% by weight to 75% by weight hemopexin, based on the total weight of all proteins in the composition.

5. (canceled)

6. The method of claim 1, wherein the composition further comprises from 1% by weight to 70% by weight albumin, based on the total weight of all proteins in the composition.

7. (canceled)

8. The method of claim 6, wherein the albumin comprises monomeric albumin, polymeric albumin, or a combination thereon.

9. The method of claim 1, wherein the composition further comprises an additional protein selected from vitamin-D binding protein, ceruloplasmin, or a combination thereof.

10. (canceled)

11. A method of treating hemorrhagic shock in a subject in thereof comprising administering a therapeutically effective amount of a protein composition comprising:
from 5% by weight to 15% by weight haptoglobin, based on the total weight of all proteins in the composition;
from 30% by weight to 50% by weight albumin, based on the total weight of all proteins in the composition;
from 1% by weight to 10% by weight hemopexin, based on the total weight of all proteins in the composition; and
from 30% by weight to 40% by weight transferrin, based on the total weight of all proteins in the composition.

12. (canceled)

13. The method of claim 1, wherein the haptoglobin has an average molecular weight of from 80 kDa to 1,000 kDa.

14. (canceled)

15. The method of claim 1, wherein the composition is substantially free of immunogenic proteins.

16. (canceled)

17. (canceled)

18. The method of claim 1, wherein the plasma protein imbalance or depletion is caused by hemorrhagic shock.

19. (canceled)

20. (canceled)

21. (canceled)

22. The method of claim 1, wherein the plasma protein imbalance or depletion is caused by a burn, surgery, organ transplantation, a hypovolemic state or hypervolemic state, sepsis, trauma, subcutaneous trauma, kidney dialysis, traumatic brain injury, traumatic brain injury combined with hemorrhagic shock, a coagulation disorder, a genetic disorder.

23-29. (canceled)

30. The method of claim 1, wherein the plasma protein imbalance or depletion requires a plasma expander or blood volume replacement.

31-34. (canceled)

35. The method of claim 1, wherein the plasma protein imbalances or depletion is prophylactically treated.

36. The method of claim 1, wherein the composition is used as an extracorporeal priming fluid or a plasma substitute for an artificial blood substitute.

37. (canceled)

38. (canceled)

39. (canceled)

40. (canceled)

41. (canceled)

42. The method of claim 1, further comprising an active agent non-covalently or covalently associated with a protein in the protein composition.

43. (canceled)

44. (canceled)

45. The method of claim 42, wherein the active agent is covalently bound to albumin or transferrin.

46. (canceled)

47. (canceled)

48. The method of claim 1, wherein an active agent is administered to the subject in combination with the protein composition.

49. (canceled)

50. (canceled)

51. A method for treating generalized inflammation in a subject in need thereof comprising administering to the subject a therapeutically effective amount of a protein composition comprising:
from 5% by weight to 99% by weight haptoglobin, based on the total weight of all proteins in the composition; and
from 1% by weight to 95% by weight transferrin, based on the total weight of all proteins in the composition.

52. A method for treating generalized inflammation in a subject in need thereof comprising administering to the subject a therapeutically effective amount of a protein composition comprising:
from 5% by weight to 15% by weight haptoglobin, based on the total weight of all proteins in the composition;
from 30% by weight to 50% by weight albumin, based on the total weight of all proteins in the composition;
from 1% by weight to 10% by weight hemopexin, based on the total weight of all proteins in the composition; and
from 30% by weight to 40% by weight transferrin, based on the total weight of all proteins in the composition.

53-59. (canceled)
